(19) United States
(12) Patent Application Publication
(10) Pub. No.: US 2011/0038791 A1
(43) Pub. Date: Feb. 17, 2011

(54) METHODS FOR INHIBITING SIX1 AND EYA PROTEINS

(76) Inventors: Heide L. Ford, Denver, CO (US); Rui Zhao, Denver, CO (US); Aaron Patrick, Denver, CO (US)

Correspondence Address: FULBRIGHT & JAWORSKI L.L.P., 600 CONGRESS AVE., SUITE 2400, AUSTIN, TX, 78701, US

(21) Appl. No.: 12/918,945

(22) PCT Filed: Feb. 25, 2009
(86) PCT No.: PCT/US09/35150

§ 371 Date: Nov. 3, 2010

Related U.S. Application Data
(60) Provisional application No. 61/031,297, filed on Feb. 25, 2008.

(51) Int. Cl. A61K 51/10; A61K 31/7088; A61K 38/00; A61K 38/16; G01N 33/53; C12N 5/071; A61K 39/395; A61K 33/24; A61P 35/00; A61P 35/02

(52) U.S. Cl. 424/1.49; 514/44 R; 514/19.3; 514/19.4; 514/19.5; 514/19.6; 435/7.21; 435/325; 424/158.1; 424/649

(57) ABSTRACT

In some aspects, the invention provides a method of inhibiting interaction of Six1 with Eya2 comprising contacting a cell expressing Six1 and Eya2 with an agent that inhibits binding of Six1 to Eya2 or an agent that inhibits the phostphatase activity of Eya2 on Six1. In other apsects, the invention provides a method of treating cancer in a subject comprising administering to said subject an effective amount of an agent that inhibits Six1 interaction with Eya2 or an agent that inhibits the phostphatase activity of Eya2 on Six1. In some embodiments, the invention further provides for the administration of a second cancer therapy to the patient. In still further aspects, the invention provides a method of identifying a candidate anti-cancer agent. The candidate inhibitor may be a nucleic acid, a protein, a peptide or a small molecule.

This application claims benefit of priority to U.S. Provisional Application Ser. No. 61/031,297, filed Feb. 25, 2008, the entire contents of which are hereby incorporated by reference.

This invention was made with government support under Grant No. CA095277 awarded by the National Institutes of Health. The government has certain rights in the invention.

### BACKGROUND OF THE INVENTION

I. Field of the Invention

The present invention relates generally to the fields of genetics, biochemistry, molecular biology, and medicine. More particularly, the invention relates to the inhibition of Six1 and Eya2.

II. Description of Related Art

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. Thus there is a need for treatments that inhibit the expression of such genes after organ development is completed.

### SUMMARY OF THE INVENTION

In one aspect, the invention provides a method of inhibiting Eya phosphatase activity comprising contacting a cell with an agent that inhibits Eya phosphatase activity. In another aspect, the invention provides a method of inhibiting interaction of Six1 with Eya comprising contacting a cell expressing Six1 and Eya with an agent that inhibits binding of Six1 to Eya. In other aspects, the invention provides a method of inhibiting interaction of Six1 with Eya interaction comprising contacting a cell with an agent that inhibits the phosphatase activity of Eya on Six1. The Eya could be Eya1, Eya2, Eya3 or Eya4. In particular aspects, the Eya is Eya2. In still other aspects, the invention provides for a method of modulating a Six1/DNA interaction comprising contacting a cell expressing Six1 with an agent that inhibits the interaction of Six1 with DNA.

A Six1 or Eya inhibitor refers to a substance that inhibits Six1 or Eya activity or expression. The inhibitor may be a nucleic acid, a protein, a peptide or a small molecule. In some embodiments, the inhibitor is a nucleic acid that encodes an antibody that binds Six1 or Eya. In other embodiments, the inhibitor is a Six1 peptide decoy that binds Eya. In still further embodiments, the inhibitor may be an Eya peptide decoy that binds Six1. In other embodiments, the Eya inhibitor may inhibit the phosphatase activity of Eya. In some embodiments, the inhibitor of the Eya2 phosphatase activity is a non-selective inhibitor. Particular non-selective inhibitors include, but are not limited to, Na₂MoO₄, β-glycerophosphate, NaF, or Na₃VO₄. In other embodiments, the inhibitor of the Eya2 phosphatase activity is a selective inhibitor. In other embodiments, the Six1 inhibitor is an agent that inhibits the Six1/DNA interaction.

In other apsects, the invention provides a method of treating cancer in a subject comprising administering to said subject an effective amount of an inhibitor of Eya phosphatase activity. The Eya could be Eya1, Eya2, Eya3 or Eya4. In particular aspects, the Eya is Eya2. In still further aspects, the invention provides a method of treating cancer in a subject comprising administering to said subject an effective amount of an inhibitor of Six1 interaction with Eya. In further aspects, the invention provides a method of treating cancer in a subject comprising administering to said subject an effective amount of an agent that inhibits the phosphatase activity of Eya on Six1. The inhibitor may be administered in any manner. A variety of such methods are well known to those of skill in the art, and include administration topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage.

In some embodiments, the invention further provides for the administration of a second cancer therapy to the patient. The second cancer therapy may be any treatment known to those of skill in the art. Non-limiting examples include radiotherapy, immunotherapy, chemotherapy, hormonal therapy or gene therapy. The current invention may be used to treat any cancer that involves Six1 and/or Eya. The cancer to be treated may be breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia. In a particular embodiment, the cancer to be treated may be breast cancer.

In still further aspects, the invention provides a method of identifying a candidate anti-cancer agent comprising contacting a cell expressing Eya with a test substance; and assessing the effect of the test substance on Eya2 phosphatase activity, wherein inhibition of the interaction of Eya2 phosphatase activity indicates that said test substance is a candidate anti-cancer agent. In yet further aspects, the invention provides a method of identifying a candidate anti-cancer agent comprising contacting a cell expressing Six1 and Eya with a test substance; and assessing the effect of the test substance on the interaction Six1 with Eya, wherein inhibition of the interaction of Six1 with Eya indicates that said test substance is a candidate anti-cancer agent. Other embodiments of the current invention provide a method of identifying a candidate anti-cancer agent comprising contacting a cell expressing Six1 and Eya with a test substance; and assessing the effect of the test substance on the phosphatase activity of Eya on Six1, wherein inhibition of the phosphatase activity of Eya on Six1 indicates that said test substance is a candidate anti-cancer agent. The candidate inhibitor may be a nucleic acid, a protein, a peptide or a small molecule.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1(A-F): Over-expression of Six1 transforms mammary epithelial cells and induces formation of aggressive breast carcinomas in a nude mouse model. (FIG. 1A) Top panel—Six1 protein levels in three MCF12A control transfected clones (Con) and in three MCF12A clones transfected with Six1 (Six1). (FIG. 1B) Soft agar assays performed on the three control and three Six1 over-expressing MCF12A clones. (FIG. 1C) Six1-dependent tumor formation in nude mice. Left mammary gland injected with MCF12A-Six1, right mammary gland injected with MCF12A-control. 15/15 mammary glands injected with Six1-overexpressing MCF12A obtained tumors, whereas 0/15 mammary glands injected with CAT-over-expressing MCF12A clones obtained tumors. (FIG. 1D) Representative tumor showing minimal tubule formation (arrow), numerous mitoses (eg: arrowheads), and marked cytologic pleomorphism (400× magnification). (FIG. 1E) Local invasion of surrounding fibroadipose tissue (with entrapment of adipocytes evident; arrow) and an adjacent normal mammary duct (arrowheads) by tumor (100× magnification). (FIG. 1F) Tumor invading a peritumoral lymphatic vessel (arrow; 200× magnification).

FIGS. 2(A-F): Fluorescently tagged Six1-overexpressing MCF7 cells form tumors that undergo metastatic spread. Gross appearance (dissecting fluorescence microscopy) and corresponding histology (H+E stained sections) of: (FIGS. 2A and D) primary tumor mass growing in the #4 mammary gland of a nude mouse (10×10⁶ cells injected); (FIGS. 2B and E) MCF7-Six1 tumor cells spreading through abdominal lymphatic vessels; (FIGS. 2C and F) MCF7-Six1 metastatic tumor in bone (leg). Tumors of the same size were formed in MCF7-CAT control injected mice, but no metastases were observed in these mice. In contrast, 20% of mice injected orthotopically with MCF7-Six1 cells obtained distant metastases, with 10% exhibiting metastasis to the bone.

FIG. 3: Domain organization of Six and Eya family of proteins (Six1 and Eya2 as examples). SD: Six Domain. HD: Homeodomain. P/S/T: Pro/Ser/Thr rich domain. ED: Eya Domain.

FIGS. 4(A-C): Expression and purification of full-length and near full-length Six1. (FIG. 4A) SDS PAGE demonstrating Six1 after gel-filtration purification (lane 1), after ion-exchange purification (lane 2), and near full-length Six1 after gel-filtration purification (lane 3). (FIG. 4B) EMSA showing that Six1 near full-length protein binds to a 16nt oligo containing the MEF3 site. (FIG. 4C) Full-length Six1 binds to an oligonucleotide containing a Six1-binding site (TCAGG).

FIG. 5: RT-PCR analyses show that Eya2 is expressed in MCF12A cells and both Eya2 & 4 are expressed in MCF7 cells.

FIGS. 6(A-D): (FIG. 6A) Purified Eya2 ED (lane 1), near full-length Eya (lane 2) and SDS analysis of central fractions of the Six1/Eya complex eluted from a gel filtration column (lane 3). (FIG. 6B) Purified Eya2 ED has phosphatase activity. (FIGS. 6C and D) Preliminary crystals of Six1/Eya2 ED (left) and western blot analysis of crystals using anti-Six1 antibody (right).

FIG. 7: Fluorescence anisotropy of DNA alone, Six1+DNA, and GST+DNA (as a negative control) in the absence and presence of DMSO. Six1 binding to DNA but not the negative controls generated a fluorescence anisotropy signal.

Fluorescence anisotropy experiments demonstrate that Six1+ED binds to the myogenin MEF3 sites, whereas the non-specific GST protein does not. DNA alone is also shown.

FIG. 8: ELISA shows that Six1 binds to the Eya2 ED in the absence and presence of DMSO. An irrelevant protein Prp8 does not show binding.

FIG. 9: Six1 protects MCF7 mammary carcinoma cells from TRAIL-mediated cell death.

FIG. 10: Removal of Six1 sensitizes ovarian cancer cells to TRAIL-mediated cell death

FIGS. 11A-D: Six1 overexpression in breast cancer predicts shortened time to metastasis, to relapse, and poorer survival. FIG. 11A shows years of metastasis-free survival; FIG. 11B indicates years of disease-free survival; FIG. 11C indicates years of disease-specific survival; FIG. 11D indicates years of disease-free survival.

FIG. 12: Six1 overexpression predicts poor survival in advanced ovarian cancers.

FIG. 13: Presence of the Six1/Eya transcriptional complex further reduces time to metastasis and significantly decreases survival time.

FIG. 14: Overexpression of Six1 leads to an increase in stem/progenitor cells in the mammary gland. MTB-control transgenic mice in which Six1 is not expressed. TOSix1 A and D line—two different transgenic lines overexpressing Six1 in the mammary gland. Cells were sorted for CD29Hi and CD24+ populations (circled), which represent the stem/progenitor cell populations within the mammary gland.

FIG. 15: Eya2 is efficiently knocked-down in MCF7-Six1 cells. Quantitative Real time RT-PCR examining Eya2 mRNA levels in stable Eya2 shRNA and scrambled shRNA clones in both MCF7-Six1 and MCF7-CAT cell lines. Two clonal isolates were chosen for analysis from two different shRNAs targeting Eya2.

FIG. 16: Loss of Eya2 in MCF7-Six1 cells reverses Six1-induced increase in the mesenchymal marker fibronectin. Western blot analysis was performed on lysates using fibronectin and β-actin antibodies. Scram: scrambled control shRNA. shRNA1: 2 clonal isolates of shRNA1 targeting Eya2. shRNA2: 2 clonal isolates of shRNA2 targeting Eya2.

FIG. 17: Loss of Eya2 in MCF7-Six1 cells reverses Six1-increased β-catenin transcriptional activity. β-catenin transcriptional activity was tested using the TOPFLASH-luciferase reporter construct and normalized to renilla luciferase activity. Data points show the mean of two individual clones across 2 experiments and error bars represent the standard error of the mean.

FIG. 18: Z-factor analyses of the high throughput screening (HTS) assay targeting Eya's phosphatase activity.

FIG. 19: HTS of 480 compounds.

FIG. 20: Several known phosphatase inhibitors can inhibit the phosphatase activity of Eya2 Eya Domain (ED).

FIGS. 21A-D: Six1 induces increased TGF-□ signaling (FIG. 21A) Six1-expressing MCF7 cells display increased p-Smad3. Western blot of analysis was performed on whole cell lysate using antibodies against p-Smad3 and β-actin. (FIG. 21B) Six1-expressing MCF7 cells have increased nuclear p-Smad3. Western blot of analysis was performed on nuclear and cytoplasmic extracts using antibodies against p-Smad, □-tubulin (cytoplasmic marker) and histone H1 (nuclear marker). (FIG. 21C) Six1-expressing MCF7 cells show increased TGF-β responsive transcription as assessed by a luciferase reporter assay using the 3TP construct and normalized to Renilla luciferase activity. P values represent statistical analysis using a paired t test (FIG. 21D) Six1-expressing MCF7 cells show increased TGF-β responsive transcription after treatment with various concentrations of TGF-β. Luciferase activity of the reporter construct 3TP was determined after treatment with the indicated concentration of TGF-β under serum-free conditions. Samples were normalized to Renilla luciferase. Data points represent the mean of three individual clones and error bars represent the standard deviation.

FIG. 22: Eya2 shRNA efficiently knocks down Eya2 in MCF7 Six1 cells, leading to a decrease in cyclin A1 levels of mRNAs determined by qRT-PCR.

FIGS. 23A-C: (FIG. 23A) Six1-overexpressing (and control-transfected) MCF12A cells in growth factor-reduced Matrigel were injected into the mammary fat pad, between the #4 and #5 nipples of 8-wk-old female nude mice. Shown are two mice in which control (Ctrl) cells were injected into the right mammary fat pad, and Six1-overexpressing cells were injected into the left mammary fat pad. Tumors arose in all cases when Six1-overexpressing cells were injected but in no cases when control cells were injected. (FIG. 23B) The weight of Six1-induced tumors is shown for each clonal isolate injected into the mammary fat pads of nude mice. Five mice were injected for each of three clonal isolates. For isolate Six1-3, one mouse bearing a Six1-3 tumor died before completion of the study, and thus, the tumor weight from this mouse is not included. Weights of all other tumors were taken 12 wk post-injection. (FIG. 23C) The MCF12A-Six1-injected cells form tumors that resemble high-grade infiltrating ductal carcinoma, characterized by poor differentiation, marked nuclear pleomorphism, and high mitotic activity. H&E-stained sections of the 12A-Six1 tumors show a high mitotic index (b; arrows in highlight, mitotic cells), and local invasion of a mammary epithelial duct (c; arrowheads), and surrounding fibroadipose tissue (c; *). These tumors exhibited invasion of a peritumoral lymphatic vessels by 12A-Six1 tumor cells (d; *, tumor; arrow, tumor in peritumoral lymphatic vessel; ˜, adjacent nontumor tissue; a and c, ×10; b, ×40; d, ×20).

FIG. 24: Six1-driven tumors manifest histologically diverse phenotypes. Shown are representative images of H&E stained tumor sections demonstrating various histological patterns of tumors observed in Six1-expressing animals.

FIGS. 25A-B: Six1 is overexpressed in MCF7 tumors in vivo. (FIG. 25A) Immunoprecipation followed by western blot analysis shows Six1 expression in 3 CAT and Six1 MCF7 transfected clonal isolates. (FIG. 25B) Representative tumor from one MCF7-CAT and MCF7-Six1 tumor stained with an Atlas Anti-Six1 antibody.

FIGS. 26A-F: Six1 over-expression induces metastasis in an orthotopic xenograft model. (FIGS. 26A-D) MCF7-Six1 tumors exhibited distant metastases to lymph nodes and bone, visible with whole body imaging of ZsGreen fluorescence (a-d) and confirmed by histology (H&E stain; a′-d′). (a and a′) Representative primary tumor, showing a poorly differentiated carcinoma. (b and b′) Tumor cells within lymphatic vessels distant from the primary tumor. (c, c′ and c″) Tumor deposits within distant lymph nodes (black arrows: tumor cells in subcapsular sinus [c′] and subcapsular lymphoid tissue [c″]). (d and d′) Large axillary metastasis, consistent with lymph node replaced by tumor. (e and e′) Bone metastasis in the femur of one mouse. (FIG. 26F) Six1 expression induced gross metastasis in 40% of tumors, while none of the control tumors metastasized. All of the mice with metastases had lesions consistent with lymph node involvement by gross analysis and/or histologic analysis; while one mouse also had a lesion within a distant lymphatic vessel and another also had bone metastasis.

### DESCRIPTION OF ILLUSTRATIVE EMBODIMENTS

### I. The Present Invention

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. The homeobox gene and transcription factor Six1 plays a critical role in the development of numerous organs through its ability to increase proliferation and decrease apoptosis, leading to an expansion of progenitor cell populations (Kawakami et al., 2000; Xu et al., 2003; Zheng et al., 2003; Laclef et al., 2003a; Laclef et al., 2003b; Ozaki et al., 2004). Six1 expression is undetectable or low in normal adult breast tissue, but it is over-expressed in 50% primary breast tumors and 90% metastatic lesions (Coletta et al., 2004; Reichenberger et al., 2005). Six1 over-expression is linked to enhanced cellular proliferation, transformation, increased tumor volume, epithelial to mesenchymal transition, and metastasis in breast cancer. (Coletta et al., 2004) (and Coletta et al., 2008). Further, epithelial-to-mesenchymal transition (EMT) is heavily associated with metastasis. The relocalization of Ecad and β-catenin is associated with EMT.

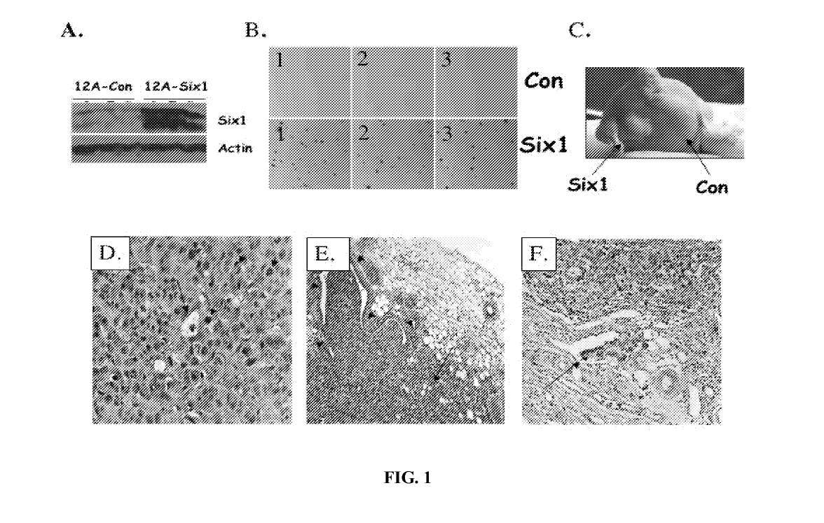

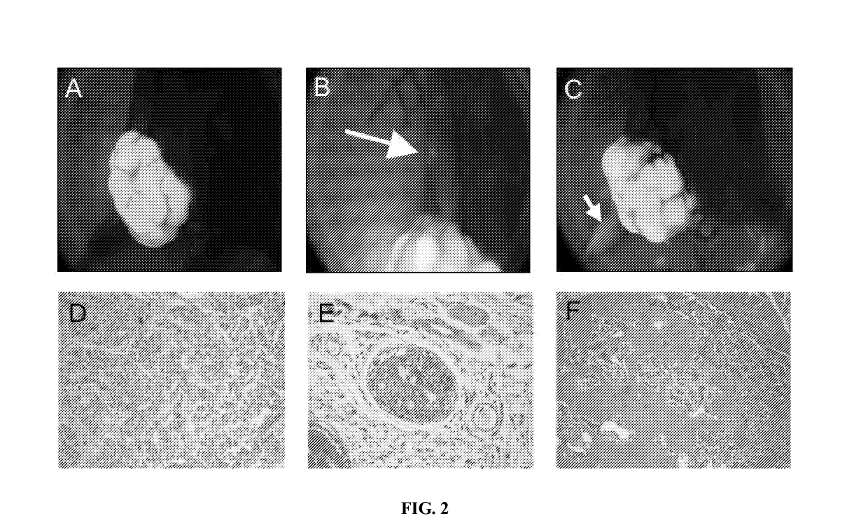

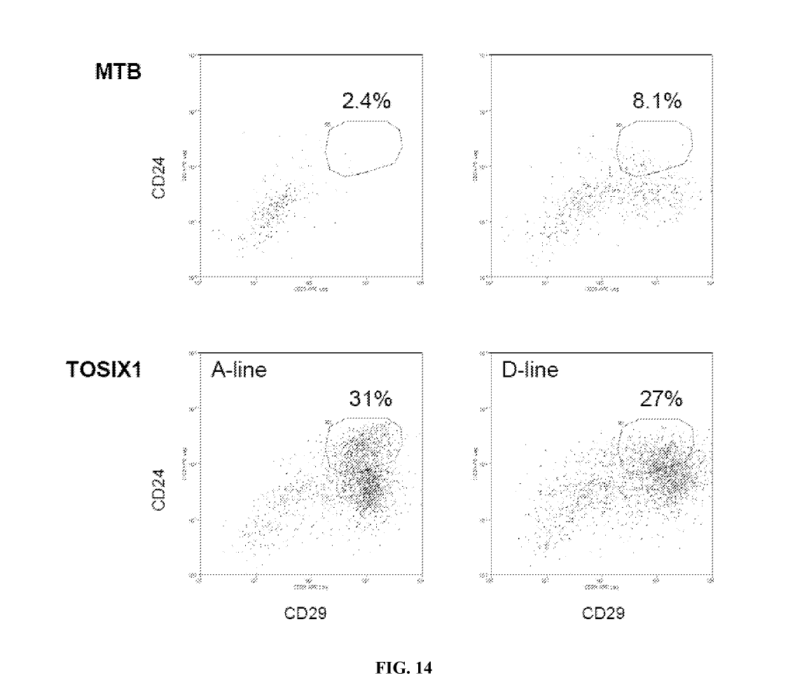

Data shows that overexpression of Six1 can transform immortalized, but otherwise normal, mammary epithelial cells, leading to highly aggressive tumors in vivo. Furthermore, data in which Six1 was overexpressed specifically in the mammary gland show that Six1 induces hyperplasia and tumor formation in vivo (FIG. 1). Together, these data indicated that Six1 is involved in tumor initiation. The higher percentage of Six1 over-expression in metastatic breast lesions indicates that it may also play an important role in breast tumor metastasis. Examination of more than 130 breast cancers demonstrates that Six1 overexpression correlates significantly with positive lymph node status (p<0.05). Six1 overexpression in tumorigenic, but nonmetastatic MCF7 cells leads to both lymphatic and bone metastasis in a mouse orthotopic breast cancer model (FIG. 2). Six1 expression leads to expansion of CD24+/CD29^{hi} mammary stem/progenitor cells (FIG. 14). These studies indicate that Six1 is a powerful oncogene that can not only induce tumorigenesis, but can also cause metastasis.

Furthermore, Six1 is overexpressed in both ovarian carcinoma and hepatacellular carcinoma, and its expression correlates with worsened survival in both cancer types (Ng et al., 2006). Six1 is also overexpressed in rhabdomyosarcomas where its expression correlates with advanced tumor stage and where it is shown to be critical for metastasis (Li et al., 2002; Khan et al., 1999; Yu et al., 2004). Six1 is amplified in a small percentage (about 5%) of human breast cancers and is overexpressed in Wilms' Tumor (Li et al., 2002). These data suggest that Six1 plays a role in the progression of many tumor types. Because Six1 is overexpressed in multiple cancers, and because it is an embryonic gene whose expression is absent in most differentiated adult tissues, it is an ideal drug target whose inactivation will inhibit tumor cell proliferation, survival, and metastasis with limited side effects. Importantly, RNA interference against Six1 decreases cancer cell proliferation and metastases in several different models of cancer. Similarly, as Six1 influences multiple stages of the tumorigenic process, targeting the Six1 transcriptional complex has the therapeutic potential to inhibit breast cancer both at early and later stages of disease progression.

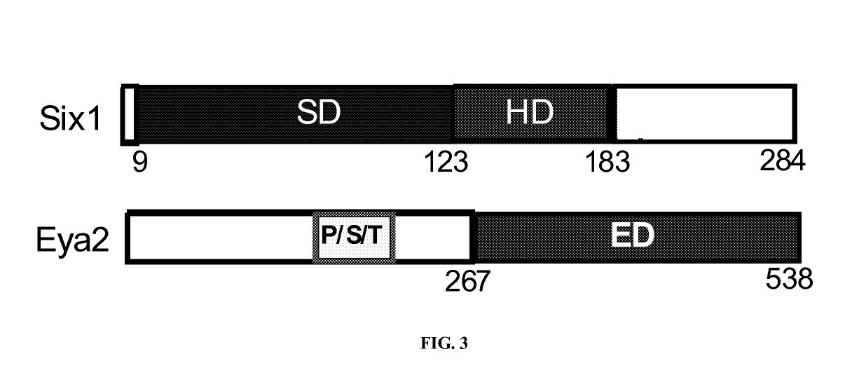

Six1 is a transcription factor with no intrinsic activation (or repression) domains, and therefore it requires co-activators to mediate its transcriptional effects. The Eya family of coactivators (Eya 1-4) are known to play important roles in Six1-mediated transcriptional activation, both in normal development (Li et al., 2003; Zhang et al., 2005) and in various diseases (Abdelhak et al., 1997; Ruf et al., 2004; Zhang et al., 2004). Both Six1 and the Eya proteins are necessary for cellular proliferation in a number of different cell types (Li et al., 2003; Zhang et al., 2005; Coletta et al., 2004), suggesting that the two proteins act together to stimulate proliferation. The Eya proteins contain a highly conserved Eya domain (ED) (Rayapureddi et al., 2003) (FIG. 3) which interacts with Six1 and has phosphatase activity (Li et al., 2003; Rayapureddi et al., 2003; Tootle et al., 2003). Recent evidence demonstrates that the Eya proteins utilize their intrinsic phosphatase activity to switch the Six1 transcriptional complex from a repressor to an activator complex (Li et al., 2003), representing the first transcription factors with intrinsic phosphatase activity that modulate transcriptional complexes (Li et al., 2003; Rayapureddi et al., 2003; Tootle et al., 2003).

In addition, since Six1 and Eya2 are developmental regulators that are expressed during embryogenesis, lost in most differentiated adult tissues, and re-expressed in tumors, therapeutic agents targeting the Six1-complex should inhibit tumor cell proliferation and metastasis with limited side effects; thus the Six1 complex is an ideal breast cancer therapeutic target.

Similarly, MEF2 is a transcription factor and developmental gene which is suppressed by HDAC (histone deacetylase) in adult heart tissue. In response to stress signals, HDAC is released leading to the activation of MEF2. Although the enzymatic domain of HDAC is not required for the suppression or activation of MEF2, inhibitors of HDAC's enzymatic activity clearly suppress MEF2 activation, illustrating the great potential of targeting enzymatic active sites (Antos et al., 2003, J. Biol. Chem. Vol. 278, p 28930).

### II. Six1 and Eya Proteins

Six1 belongs to the Six family of homeobox genes (Six1-6) encoding transcription factors that play vital roles in the development of many organs (Kawakami et al., 2000). Six1-6 share a DNA binding homeodomain (HD) and a Six domain (SD) responsible for co-activator binding (Kawakami et al., 2000). In particular, Six1 plays a role in cell growth, cell survival and cell migration during normal cell development. Six1 plays a critical role in the onset and progression of a significant proportion of breast and other cancers, but has never before been clinically targeted. The Six1 homeobox gene encodes a transcription factor that is crucial for the development of many organs but is down-regulated after organ development is complete. Its expression is low or undetectable in normal adult breast tissue but the gene is over-expressed in 50% of primary breast tumors and 90% of metastatic lesions. Examination of public microarray databases containing more than 535 breast cancer samples demonstrates that Six1 levels correlate significantly with shortened time to relapse, shortened time to metastasis, and decreased overall survival. In addition, Six1 overexpression correlates with adverse outcomes in numerous other cancers, including ovarian, hepatocellular carcinoma, and rhabdomyosarcoma. Using mouse models of mammary cancer, it was recently demonstrated that over-expression of Six1 results in enhanced proliferation, transformation, increased tumor volume, and metastasis. Importantly, RNA interference against Six1 decreases cancer cell proliferation and metastases in several different cancer models.

Six1 was shown to bind tightly to the MEF3 motif (TCAGGTT) (Spitz et al., 1998). This sequence is different from the TAAT core sequence bound by the canonical HD, likely due to the fact that the HD in Six1 differs from the “classic” HD at two highly conserved residues contacting DNA. (FIG. 3). The Six type HD is believed to confer a unique DNA binding specificity to the Six family members that differs from the TAAT core in the classic HD. However, the consensus Six1 recognition sequence remains unknown. A limited number of potential Six1 targets are identified (Kawakami et al., 1996; Spitz et al., 1998; Ando et al., 2005) and, indeed, none of them contain the TAAT core. Interestingly, these targets do not share an obvious consensus sequence, possibly due to the limited number of sequences analyzed. The Six1 target most relevant to breast tumorigenesis is the cyclin A1 promoter (Coletta et al., 2004). The transcriptional up-regulation of cyclin A1 by Six1 leads to an increase in proliferation in mammary carcinoma cells and Six1 mediated cell cycle progression is dependent on cyclin A1 (Coletta et al., 2004). In addition to the HD, the Six family members contain a conserved and novel Six-domain (SD) (FIG. 1) (Oliver et al., 1995). The SD contributes to DNA binding as well as to protein interaction with cofactors (Kawakami et al., 2000; Oliver et al., 1995).

Six1 does not have an intrinsic activation or repression domain and requires the Eya coactivator proteins to activate transcription. The Eya proteins utilize their intrinsic phosphatase activity to switch the Six1 transcriptional complex from a repressor to an activator complex. The Six1-Eya interaction is essential for proliferation during embryonic development, and both Six1 and Eya2 have been independently implicated in the same types of cancer. Because the Eya co-activator contains a unique protein phosphatase domain whose activity is required to activate Six1, it may serve as a novel anti-cancer drug target.

The Eya proteins are mammalian homologues of the Drosophila eyes absent genes. Four family members exist in mammals, Eya1-4, each which contain a divergent N-terminus, an internal proline-serine-threonine (PST) rich activation domain, and a highly conserved C-terminal Eya domain (ED) (Rayapureddi et al., 2003). The ED is responsible for interactions with the SD of the Six family of proteins. The ED contains signature motifs of the haloacid dehalogenase (HAD) hydrolases, a diverse collection of enzymes including magnesium-dependent phosphatases (Rayapureddi et al., 2003; Tootle et al., 2003; Li et al., 2003). The encoded Eya1 protein may play a role in the developing kidney, branchial arches, eye, and ear. Mutations of this gene have been associated with branchiootorenal dysplasia syndrome, branchiootic syndrome, and sporadic cases of congenital cataracts and ocular anterior segment anomalies. Four transcript variants encoding three distinct isoforms have been identified for the Eya1 gene. The encoded Eya2 protein may be post-translationally modified and may play a role in eye development. Five transcript variants encoding three distinct isoforms have been identified for the Eya2 gene. The encoded Eya3 protein may act as a transcriptional activator and have a role during development. A similar protein in mice acts as a transcriptional activator. The encoded Eya4 protein may act as a transciptional activator and be important for continued function of the mature organ of Corti. Mutations in this gene are associated with postlingual, progressive, autosomal dominant hearing loss at the deafness, autosomal dominant nonsyndromic sensorineural 10 locus. Three transcript variants encoding distinct isoforms have been identified for the Eya4 gene.

Recent evidence demonstrates that the Eya proteins utilize their intrinsic phosphatase activity to switch the Six1 transcriptional complex from a repressor to an activator complex (Li et al., 2003). Although phosphorylation events often affect transcriptional activity, the Eya proteins represent the first transcription factors with intrinsic phosphatase activity that modulates transcriptional complexes. Eya proteins are considered the founding members of a new class of non-thiol based protein phosphatases since no other HAD phosphatases have protein phosphatase activity and most protein phosphatases use a catalytic Cys instead of the Asp used by Eya proteins (Tootle et al., 2003).

Since Six1 does not contain any intrinsic activation/suppression domain, it relies on other cofactors such as Eya for its transcriptional activity. Eya knockout mice phenocopy Six1 knockout mice (Xu et al., 1999). Six1's activity on cellular proliferation was also found to be dependent on Eya (Li et al., 2003). As Six1 contributes to breast tumorigenesis by stimulating cellular proliferation, the interaction between Eya and Six1 may be critical for Six1-mediated tumorigenesis. In addition, Eya2 is found to be amplified in ovarian (Zhang et al., 2005) and pancreatic cancers. Its over-expression, like Six1, is associated with increased proliferation and shortened overall survival of advanced ovarian cancer patients (Zhang et al., 2005). FIG. 16 demonstrates that loss of Eya2 in MCF7 cells reverses the ability of Six1 to induce a more mesenchymal phenotype in MCF7 cells, as assessed by the levels of the fibronectin protein. Similarly, the relocalization of E-cad and β-catenin is associated with EMT. Thus, a reversal of Six1 induced EMT suggests that Eya2 loss may reverse the metastatic phenotype caused by Six1.

A. Functional Aspects

When the present application refers to the function or activity of Six1, it is meant that the molecule in question has the ability to activate Eya. Determination of which molecules possess this activity may be achieved using assays familiar to those of skill in the art. For example, transfer of genes encoding products that inhibit the activation of Six1, or variants thereof, into cells that have a functional Six1 product will identify, by virtue of an increased level of apoptosis, those molecules having a Six1 or Eya2 inhibitor function. An endogenous Six1 or Eya polypeptide refers to the polypeptide encoded by the cell's genomic DNA.

On the other hand, when the present invention refers to the function or activity of a Six1 or Eya inhibitor, one of ordinary skill in the art would further understand that this includes, for example, the ability to specifically or competitively bind Six1 or an ability to reduce or inhibit its activity, such as reduce its ability to bind Eya. Thus, it is specifically contemplated that a Six1 modulator may be a molecule that affects Six1 expression, such as by binding a Six1-encoding transcript. Determination of which molecules are suitable modulators of Six1 or Eya may be achieved using assays familiar to those of skill in the art.

B. Peptides

Inhibitors of Six1 or Eya may be peptides. Peptides of the current invention will comprise molecules of 5 to no more than about 50 residues in length. A particular length may be less than 39 residues, less than 35 residues, less than 30 residues, less than 25 residues, less than 20 residues, less than 15 residues, or less than 13, including 5, 6, 7, 8, 9, 10, 11 or 12 residues, and ranges of 5-11 residues, 5-15 residues, 5-20 residues, 5-25 residues, 5-30 residues, 5-35 residues, 5-38 residues, or 5-40 residues. The peptides may be generated synthetically or by recombinant techniques, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration), as described in further detail below.

The peptides may be labeled using various molecules, such as fluorescent, chromogenic or colorimetric agents. The peptides may also be linked to other molecules, including other anti-cancer agents. The links may be direct or through distinct linker molecules. The linker molecules in turn may be subject, in vivo, to cleavage, thereby releasing the agent from the peptide. Peptides may also be rendered multimeric by linking to larger, and possibly inert, carrier molecules.

C. Variants of Six1 or Eya

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below.

In making substitutional variants, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of Six1 or Eya, but with altered and even improved characteristics.

D. Purification of Proteins/Peptides

It may be desirable to purify Six1 or Eya or fragments or inhibitors thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

E. Synthesis

The peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### III. Nucleic Acid Vectors

In particular embodiments, the inhibitor or candidate substance of the present invention may be an isolated nucleic acid or a recombinant vector the invention concerns isolated DNA segments and recombinant vectors. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated Six1 or Eya polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular gene, such as the human Six1 or Eya gene. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides, for example, a modified gelonin toxin. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (Sambrook et al., 1989; Ausubel et al., 1996, both incorporated herein by reference). In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

A. Expression Vectors or Expression Cassettes

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. The identity of tissue-specific promoters or elements, as well as assays to characterize their activity, is well known to those of skill in the art.

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5□-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (See Chandler et al., 1997, incorporated herein by reference).

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Particular embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (available on the world wide web at atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

E. Methods of Gene Transfer

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### IV. Screening Methods

In some embodiments, the present invention provides a method of screening for a Six1 or Eya modulator. Compounds may be screened to find modulators that could inhibit Six1 binding with Eya, or inhibit the phostphatase activity of Eya on Six1. In one embodiment, binding affinity assays and E3 liagase enzyme acitivity assays may be used for determining inhibitor effeciency. One of skill in the art would be aware that there are several methods available, including but not limited to those described below.

A. Screening for Modulators of Six1 or Eya

The present invention further comprises methods for identifying modulators of Six1 or Eya activity. These assays may comprise random screening of large libraries of candidate substances; alternatively, the assays may be used to focus on particular classes of compounds selected with an eye towards structural attributes that are believed to make them more likely to modulate the function of Six1 or Eya.

By function, it is meant that one may assay for a measurable effect on Six1 or Eya activity, binding activity or inhibition of the phostphatase activity of Eya on Six1. To identify a Six1 or Eya modulator, one generally will determine the activity or level of inhibition or modulation of Six1 or Eya in the presence and absence of the candidate substance, wherein a modulator is defined as any substance that alters these characteristics. For example, a method generally comprises providing a candidate modulator; admixing the candidate modulator with an isolated protein or cell expressing the protein; measuring one or more characteristics of the protein or cell; and comparing the characteristic measured with the characteristic of the protein or cell in the absence of said candidate modulator, wherein a difference between the measured characteristics indicates that said candidate modulator is, indeed, a modulator of the protein or cell. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may be a “modulator” of Six1 or Eya, i.e., potentially affect Six1 or Eya Six1 or Eya activity, binding activity or inhibition of the phostphatase activity of Eya on Six1, directly or indirectly. A modulator may be a “Six1 or Eya inhibitor,” which is a compound that overall effects an inhibition of Six1 or Eya activity, which may be accomplished by inhibiting Six1 or Eya expression, translocation or transport, function, expression, post-translational modification, location, half-life, or more directly by preventing its activity, such as by binding Six1 or Eya.

The candidate substance may be a protein or fragment thereof, a small molecule, or even a nucleic acid molecule. An example of pharmacological compounds will be compounds that are structurally related to Six1 or Eya, or a molecule that binds Six1 such as Eya. In some embodiments, the crystal structure of Six1 and/or an Eya protein may be used to develop small molecule inhibitors that disrupt Six1-DNA or Six1-Eya interactions or Eya phosphatase activity. Two previous papers have reported that the phosphatase activity of mouse Eya3 Eya Domain can be inhibited by several known phosphatase inhibitors. (Rayapureddi et al., 2003; Tootle et al., 2003). Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single-chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor or activator according to the present invention may be one which exerts its inhibitory or activating effect upstream, downstream or directly on Six1 or Eya. Regardless of the type of inhibitor or activator identified by the present screening methods, the effect of the inhibition or activator by such a compound results in alteration in Six1 or Eya activity as compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. Screening for Six1 or Eya Activity

In some embodiments of the present invention, methods of assaying whether the candidate inhibits Six1 or Eya activity. These methods may involve screening for the activity of Six1 or Eya. Six1 or Eya activity may be evaluated using any of the methods and compositions disclosed herein, including assays involving evaluating Six1's or Eya's binding activity, inhibition of the phostphatase activity of Eya on Six1, or Six1's ability to inhibit apoptosis. Any other the compounds or methods described herein may be employed to implement these methods.

Assays to evaluate the level of expression of a polypeptide are well known to those of skill in the art. This can be accomplished also by assaying Six1 or Eya mRNA levels, mRNA stability or turnover, as well as protein expression levels. It is further contemplated that any post-translational processing of Six1 or Eya may also be evaluated, as well as whether it is being localized or regulated properly. In some cases an antibody that specifically binds Six1 or Eya may be used.

Furthermore, it is contemplated that the status of the gene may be evaluated directly or indirectly, by evaluating genomic DNA sequence comprising the Six1 or Eya coding regions and noncoding regions (introns, and upstream and downstream sequences) or mRNA sequence. The invention also includes determining whether any polymorphisms exist in Six1 or Eya genomic sequences (coding and noncoding). Such assays may involve polynucleotide regions that are identical or complementary to Six1 or Eya genomic sequences, such as primers and probes described herein.

In particular embodiments, Six1 is detected by IHC on paraffin-embedded, formalin fixed tissue. In other embodiments, Six1 is detected by selective reactive monitoring mass spectrometry.

### V. Six1 or Eya Detection Methods

It is within the general scope of the present invention to provide methods for the detection of proteins and mRNA. Any method of detection known to one of skill in the art falls within the general scope of the present invention.

A. Protein Detection

In certain embodiments, the present invention concerns determining the expression level of the protein Six1 or Eya. In other embodiments, the invention provides for an inhibitor of Six1 or Eya, wherein the inhibitor may be a protein. As used herein, a “protein,” “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the proteinaceous composition may comprise at least one antibody, for example, a Six1 or Eya. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (see, e.g., Harlow et al., 1988; incorporated herein by reference).

1. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

2. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

3. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize Six1 or Eya or to evaluate the amount Six1 or Eya in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer. Specific molecular markers are characteristic of particular cancer types.

Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

4. Antibodies

Another embodiment of the present invention are antibodies, in some cases, a Six1 or Eya antibody. It is understood that antibodies can be used for inhibiting or modulating Six1 or Eya. It is also understood that this antibody is useful for screening samples from human patients for the purpose of detecting Six1 or Eya present in the samples. The antibody also may be useful in the screening of expressed DNA segments or peptides and proteins for the discovery of related antigenic sequences. In addition, the antibody may be useful in passive immunotherapy for cancer. All such uses of the said antibody and any antigens or epitopic sequences so discovered fall within the scope of the present invention.

In certain embodiments, the present invention involves antibodies. For example, all or part of a monoclonal, single-chain, or humanized antibody may function as a modulator of Six1 or Eya. Other aspects of the invention involve administering antibodies as a form of treatment or as a diagnostic to identify or quantify a particular polypeptide, such as Six1 or Eya. As detailed above, in addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (monoclonal antibodies) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).

5. Protein Arrays

Protein array technology is discussed in detail in Pandey and Mann (2000) and MacBeath and Schreiber (2000), each of which is herein specifically incorporated by reference.

These arrays, typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as Six1 or Eya.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

B. Nucleic Acid Detection

In addition to their use in directing the expression of Six1 or Eya modulator proteins, polypeptides and/or peptides, the nucleic acid sequences disclosed herein have a variety of other uses. For example, they have utility as probes or primers for embodiments involving nucleic acid hybridization. They may be used in diagnostic or screening methods of the present invention. Detection of nucleic acids encoding Six1 or Eya or Six1 or Eya modulators are also encompassed by the invention. See WO 2004/048933. In certain embodiments, the present invention concerns determining the level of Six1 or Eya expression by determining the level of gene expression. In other embodiments, the invention provides for an inhibitor of Six1 or Eya, wherein the inhibitor may be a nucleic acid. Generally, the present invention concerns polynucleotides and oligonucleotides, isolatable from cells, that are free from total genomic DNA and that are capable of expressing all or part of a protein or polypeptide. The polynucleotides or oligonucleotides may be identical or complementary to all or part of a nucleic acid sequence encoding a Six1 or Eya amino acid sequence. These nucleic acids may be used directly or indirectly to assess, evaluate, quantify, or determine Six1 or Eya expression.

As used in this application, the term “Six1 or Eya polynucleotide” refers to a Six1 or Eya-encoding nucleic acid molecule that has been isolated essentially or substantially free of total genomic nucleic acid to permit hybridization and amplification, but is not limited to such. Therefore, a “polynucleotide encoding Six1 or Eya” refers to a DNA segment that contains wild-type, mutant, or polymorphic Six1 or Eya polypeptide-coding sequences isolated away from, or purified free from, total mammalian or human genomic DNA. A Six1 or Eya oligonucleotide refers to a nucleic acid molecule that is complementary or identical to at least 5 contiguous nucleotides of a Six1 or Eya-encoding sequence, which is the cDNA sequence encoding human Six1 or Eya.

It also is contemplated that a particular polypeptide from a given species may be represented by natural variants that have slightly different nucleic acid sequences but, nonetheless, encode the same protein.

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including such sequences from Six1 or Eya encoding sequences.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a Six1 or Eya-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of Six1 or Eya with respect to diagnostic, as well as preventative and treatment methods of the invention.

5. Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### VI. Methods of Therapy

In some embodiments, the invention provides compositions and methods for the treatment of cancer. In one embodiment, the invention provides a method of treating cancer comprising administering to a patient an effective amount of an inhibitor of the interaction of Six1 and Eya. This treatment may be further combined with additional cancer treatments. One of skill in the art will be aware of many treatments that may be combined with the methods of the present invention, some but not all of which are described below.

The present invention also involves, in another embodiment, the treatment of cancer. The types of cancer that may be treated, according to the present invention, is limited only by the involvement of Six1 or Eya. By involvement, it is not even a requirement that Six1 or Eya be mutated or abnormal—the overexpression of this tumor suppressor may actually overcome other lesions within the cell. Thus, it is contemplated that a wide variety of tumors may be treated using Six1 or Eya inhibition therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Peptide Therapy

One therapy approach is the provision, to a subject, of Six1 or Eya inhibitor polypeptide, fragments, synthetic peptides, mimetics or other analogs thereof. The protein/peptide may be produced by recombinant expression means or, if small enough, generated by an automated peptide synthesizer. Formulations would be selected based on the route of administration and purpose including, but not limited to, liposomal formulations and classic pharmaceutical preparations.

B. Antibody Therapy

Applicants also contemplate the use of antibodies to Six1 or Eya. Antibodies will be administered according to standard protocols for passive immunotherapy. Administration protocols would generally involve intratumoral, local or regional (to the tumor) administration, as well as systemic administration.

In addition, the antibody reagent may be altered, such that it will have one or more improved properties. The antibody may be recombinant, i.e., an antibody gene cloned into an expression cassette which is then introduced into a cell in which the antibody gene was not initially created. The antibody may be single-chain, a fragment (Fab, Fv, Vh, ScFv), chimeric or humanized.

C. Formulations and Routes for Administration to Patients

In some embodiments, the invention provides a method of treating cancer comprising administering to a patient an effective amount of an inhibitor of the interaction of Six1 and Eya. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

D. Cancer Combination Treatments

In some embodiments, the method further comprises treating a patient with cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis or other therapeutic intervention. It also is conceivable that more than one administration of the treatment will be desired.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Gene Therapy

In yet another embodiment, the secondary treatment is a gene therapy in which a therapeutic polynucleotide is administered before, after, or at the same time as a Six1 or Eya inhibitor is administered. Delivery of a Six1 or Eya inhibitor in conjunction with a vector encoding one of the following gene products may have a combined anti-hyperproliferative effect on target tissues. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} (4 has been biochemically characterized as a protein that specifically binds to and inhibits CDK4, and thus may regulate Rb phosphorylation (Serrano et al., 1993; Serrano et al., 1995). Since the p16^{INK4} (4 protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/Six1 or Eya2, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

F. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

G. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### VII. Examples

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of the Six1 DNA Recognition Sequence

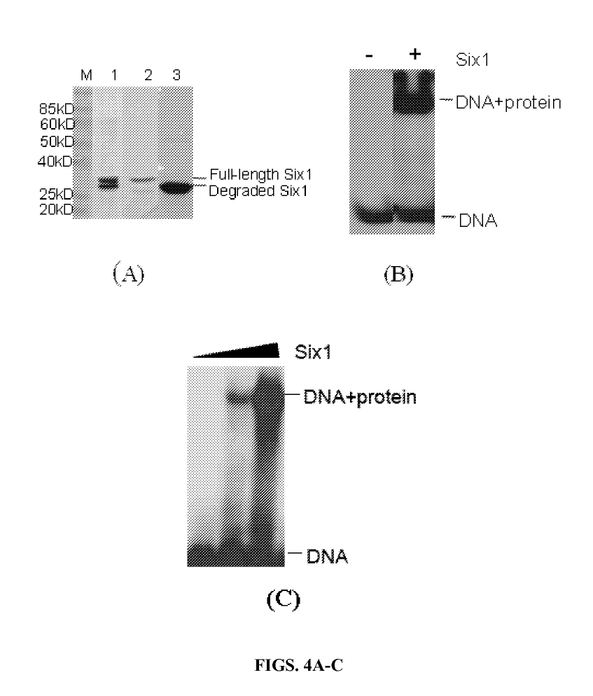

The inventors obtained purified Six1 protein by expressing Six1 as a GST-fusion protein in E. coli. GST-Six1 was first purified by glutathione resin, cleaved by thrombin to remove GST, and further purified with a gel filtration column. Gel filtration purified Six1 contains the full-length protein and a naturally-degraded form of Six1 (FIG. 4A). N-terminal sequencing and mass spectrum analysis indicate that the natural degradation occurs at residue 259, downstream of the HD. Further ion-exchange chromatography generated the pure full-length protein (FIG. 4A).

Full-length Six1 can bind to several known Six1 target promoters in an electrophoretic mobility shift assay (EMSA), for example, MEF3 (FIG. 4C). Purified Six1 binds to an 18 nt dsDNA containing the MEF3 site (a well-characterized Six1 binding site in the myogenin promoter (Spitz et al., 1998)) with a Kd of 1 μM. The addition of Eya's ED increases the binding affinity to about 300 nM (FIG. 4B).

A near full-length Six1 was expressed and purified similarly as the full-length Six1 (FIG. 4A). The truncated Six1 binds DNA similarly as the full-length protein. Six1 near full-length protein binds to a 16 nt DNA containing the MEF3 motif as demonstrated using EMSA and the preliminary estimation of the Kd is ˜1 μM (FIG. 4B). The naturally occurring degradation indicates the existence of flexible regions in the protein. Removal of the flexible regions will generate more compact proteins that are usually easier to crystallize than flexible proteins, and the inventors will therefore perform all crystallization trials with Six1 near full-length protein, which contains the conserved SD and HD (FIG. 3).

The minimum Six1 recognition sequence in the cyclin A1 promoter may be identified. The region in the cyclin A1 promoter required for Six1 activation has been mapped to −112 to −37 and Six1 is present in this region using a CHIP assay (Coletta et al. 2004). Six1 likely directly interacts with the cyclin A1 promoter since all known homeobox domains interact with DNA directly instead of through adaptor proteins. EMSA may be performed using purified Six1 and serial deletions of the cyclin A1 promoter to identify the minimum Six1 binding site. The site can be confirmed using in vivo luciferase assay by mutating this site in the context of the cyclin A1 promoter (Coletta et al. 2004).

SELEX can be used to identify the Six1 DNA recognition sequence. GST-Six1 has been shown to bind DNA specifically and the inventors will use the purified GST-Six1 to perform SELEX experiments. A DNA pool containing a random sequence core can be incubated with GST-Six1, bound to glutathione resin, washed with low salt buffer, and eluted with high salt buffer. A 12 bp random sequence core may be used since most consensus sequences recognized by transcription factors are less than 12 bp. The eluted DNA may be amplified using PCR and subjected to multiple rounds of binding, washing, and amplification. The final DNA pool may be sub-cloned into a plasmid vector and sequenced. The sequences can be aligned to reveal the consensus Six1 DNA recognition sequence.

### Example 2

### Development of a Transgenic Model of Six1 Induced Mammary Tumorigenesis

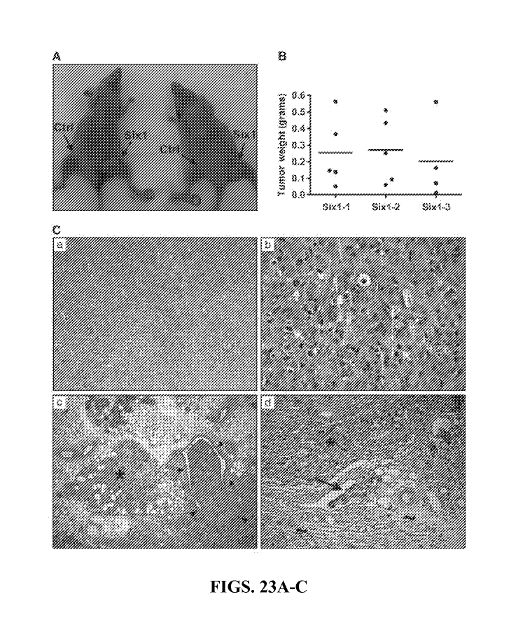

Six1 is re-expressed in a number of cancers, including breast cancer, and may promote tumor initiation and/or progression by reinstating a developmental program out of context. Over-expression of Six1 transforms immortalized, but otherwise normal, mammary epithelial cells, forming highly aggressive tumors when injected orthotopically into the mammary glands of nude mice (Coletta et al., 2008 and FIG. 23). A xenograft model is used to determine the role of Eya2 in Six1-induced tumorigenesis, and to determine whether small molecules targeting the Six1 transcriptional complex can inhibit tumor formation and growth.

An inducible transgenic mouse model of Six1 overexpression in the mammary gland was developed. To generate the transgenic model, the MTB transgenic line (Gunther et al., 2002) was crossed with transgenic mice containing HA-tagged Six1 downstream of tet operator sequences (TetSix line), resulting in bitransgenic offspring (TOSix). The MTB line expresses an MMTV-LTR driven reverse tetracycline transcriptional activator (rtTA), so that treatment of TOSix animals with doxycycline (dox) activates rtTA, which binds the tet-promoter and initiates transcription of Six1. Five TetSix founder lines were established, containing variable copy numbers of the transgene (data not shown). Two lines were fully characterized, and when both lines where crossed to MTB mice, overexpression of Six1 in the mammary gland of multiparous mice led to the development of overt mammary tumors in approximately 40% ( 9/25) of animals expressing Six1. In contrast, only 7% ( 1/14) of control animals developed a mammary tumor, and this tumor did not occur until the mouse was over two years of age. The average latency of tumor onset in the Six1-expressing animals was approximately 17.5 months after starting doxycycline or vehicle treatment, suggesting that Six likely cooperates with additional pathways to induce tumorigenesis.

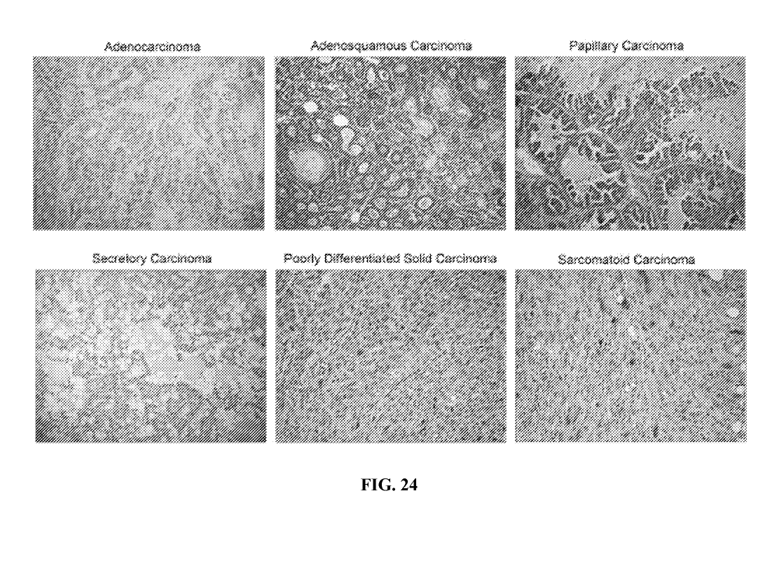

Mammary specific, inducible Six1 over-expression leads to the development of neoplastic breast lesions of diverse histologies much like human breast cancer (FIG. 24). The majority of mammary tumors arising in the Six1 transgenic mice show glandular (“adeno”) differentiation, as seen in human infiltrating ductal breast carcinoma. Two thirds of the tumors show some degree of squamous differentiation. Additional histologic patterns, which also correlate with human tumors, include secretory and papillary differentiation. Interestingly, some of the mammary tumors show high-grade, poorly differentiated, solid areas, analogous to poorly differentiated human carcinoma. Most strikingly, some tumors exhibit highly aggressive spindle cell morphology, analogous to highly aggressive human sarcomatoid carcinoma, demonstrating the induction of a clear epithelial to mesenchymal transition in vivo. Forty-three percent of mammary lesions induced by Six1 exhibit features of intraepithelial neoplasia, while 86% of mammary tumors show invasive features, characterized by loss of regular glandular architecture, stromal desmoplasia and an infiltrative growth pattern. Overall, the Six1-induced neoplastic lesions appear to have an in-situ origin, show diverse differentiation, and exhibit progression to highly aggressive malignant neoplasms, as observed in human carcinoma of the breast. This is notable, as many transgenic models of mammary cancer do not mimic human breast cancer as well as this Six1 over-expression model does. Together, these data clearly demonstrate that Six1 over-expression can induce highly aggressive breast cancer.

### Example 3

### Development of a Model of Six1-Induced Breast Cancer Metastasis

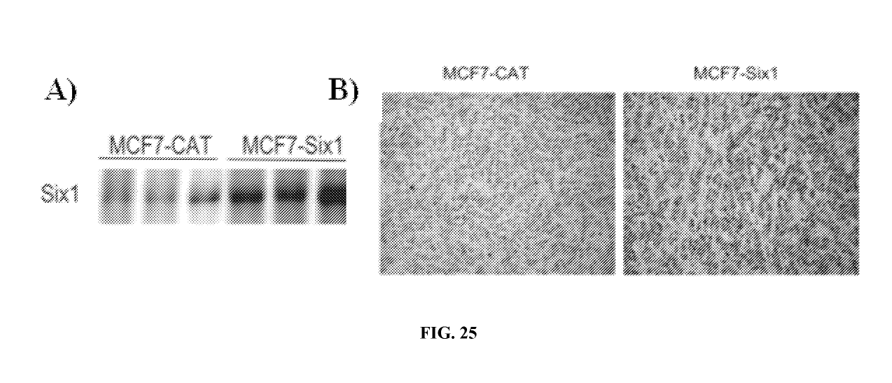

To obtain direct evidence that Six1 plays a role in breast cancer metastasis, Six1 was overexpressed in a tumorigenic, but non-metastatic breast cancer cell line (MCF7) (FIG. 25A) (Coletta et al., 2004). This is a very rigorous test because MCF7 cells are NOT inherently metastatic in nude mouse assays (Zhang et al., 1997; Kurebayashi et al., 1993; Kern et al., 1994). The derived cell lines (Ford et al., 1998) were tagged with a fluorescent marker (Zs-green) to enhance the ability to follow metastasis, particularly micrometastasis, in vivo. Three clonal MCF7-Six1 and 3 clonal MCF7-CAT lines, all stable for ZSgreen, were injected (1×10⁶ cells) into the mammary fat pad of the 4th mammary gland (following implantation of estrogen pellets into the mice) of 5 mice per line, and were traced over a period of time using the Illumatool Bright Light System LT-9900 (Lightools Research). Fluorescent tumors were observed in the live mice after 3-4 weeks. Staining for Six1 in the MCF7-CAT versus MCF7-Six1 tumors demonstrated that Six1 overexpression was maintained in the tumors in vivo (FIG. 25B).

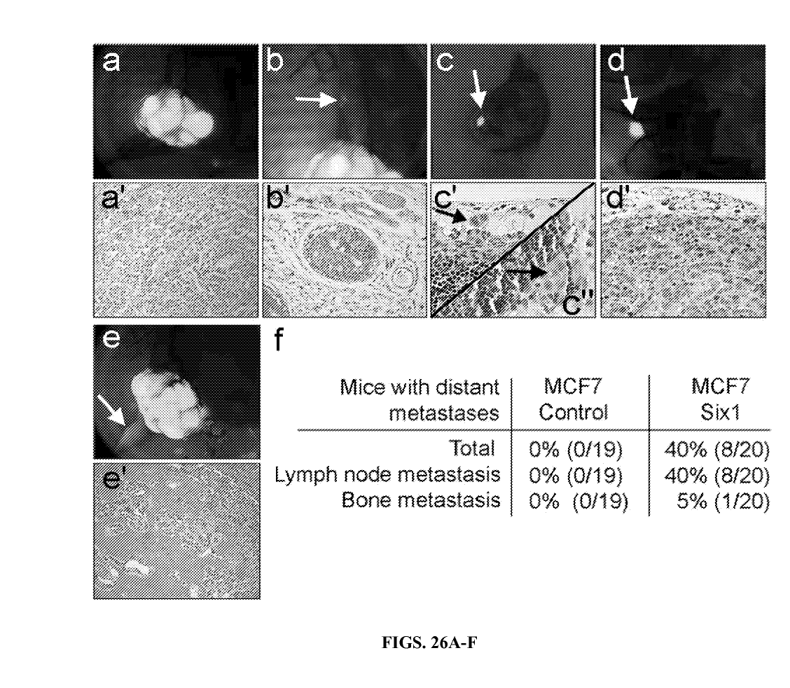

All mice were sacrificed when the tumors reached 2 cm3, at which point metastases were observed via green fluorescence in 40% of mice (8/20) injected orthotopically with MCF7-Six1 cells. No metastases were observed in mice injected orthotopically with MCF7-CAT clones (0/19). Six1-overexpressing tumors were found to metastasize to the lymphatics and lymph nodes (FIGS. 26A-D) and bone (FIG. 26E), by both green fluorescence and by subsequent histologic analyses.

When this experiment was repeated in NOD/SCID mice, which are more immunocompromised than nude mice, Six1 over-expression led to metastasis in 75% of the animals, as compared to 17% of animals injected with cells lacking Six1 (data not shown). Together, these data provide direct experimental evidence that Six1 over-expression in breast cancer cells induces metastasis of otherwise non-metastatic cells, and further demonstrate that Six1 over-expression leads to metastasis to sites relevant to human breast cancer: lymph nodes and bone. Thus, overall the studies demonstrate that Six1 can induce breast tumorigenesis and metastasis.

### Example 4

### Determine the Role of the Eya Proteins and Their Phosphatase Activity in Six1-Mediated Breast Tumorigenesis/Metastasis

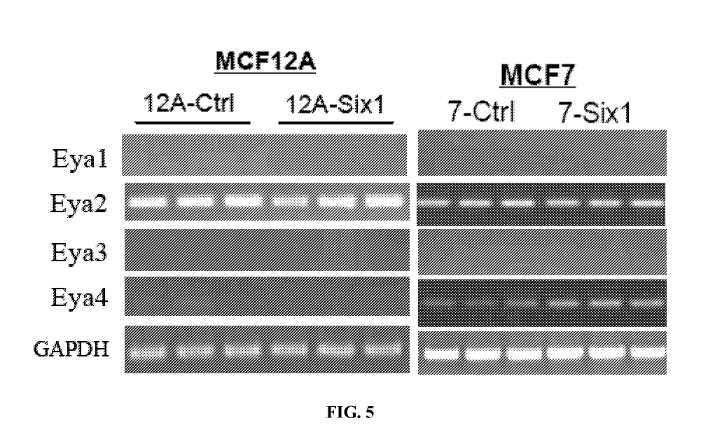

To determine whether phenotypes associated with Six1 overexpression, including increased proliferation and tumor burden, transformation (FIGS. 1A-F) and metastases (FIGS. 2A-F), are dependent on the activation of Six1 by Eya, the inventors first examined which, if any, of the 4 Eya family members are expressed in MCF12A and MCF7 cells, where these phenotypes are observed. Reverse-transcription and real time RT-PCR shows that Eya2 is the only Eya expressed in MCF12A cells, whereas Eyas 2 and 4 are both expressed in MCF7 cells (FIG. 5). However, Eya2 is more than 10,000-fold as abundant as Eya4 in MCF7 cells (data not shown). Eya2, as well as Six1, have recently been implicated in ovarian and pancreatic cancers, and Eya2 is the most prevalent Eya in both MCF12A and MCF7 cells. In addition, as outlined above, Six1 and Eya2 are coordinately overexpressed in breast cancers with poor prognosis. This suggests that Eya2 may be a relevant cofactor for Six1 in human breast cancer. However, because Eya 4 is also present in MCF7 cells and Eya4 expression increases with Six1 overexpression (FIG. 5), it is possible that Eya4 plays an additional role in breast cancer, specifically stimulating metastasis in concert with Six1.

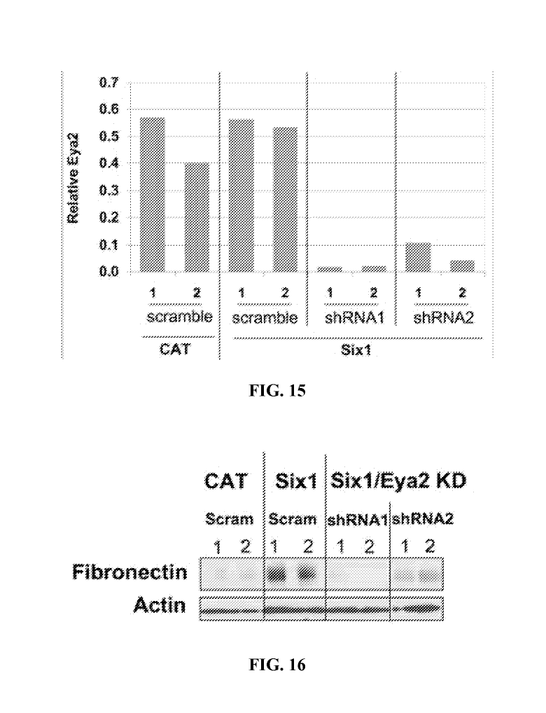

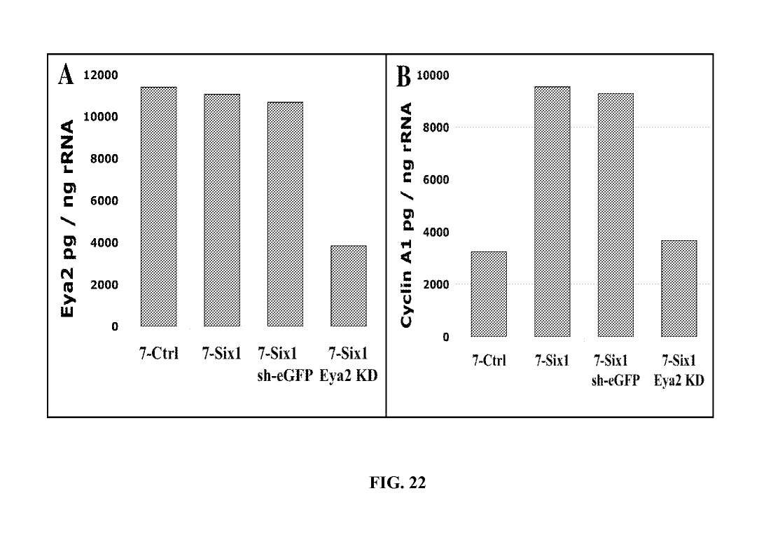

To examine whether the Eyas are required for Six1-mediated proliferation, the inventors transiently knocked down Eya2 in MCF12A cells, and Eyas 2 and 4 in MCF7 cells engineered to overexpress Six1 and assess the consequence of Eya knockdown on the Six1-induced phenotype. The inventors have previously shown that Six1 overexpression in MCF7 and MCF12A cells leads to increased levels of cyclin A1 mRNA and its kinase activity, as well as to an increase in proliferation in U.S. Publication 2006/0078903, incorporated by reference in its entirety. The inventors generated stable clonal isolates of Eya2 knockdown in MCF7 cells (FIG. 15). These stable knockdowns can be used to assess EMT in vitro (which may not be reversed in a transient setting), and in vivo, where transient knockdowns would not enable the assessment of the role of Eya2 in tumor growth/metastasis. In particular, Eya2 was knocked down in mammary carcinoma cells that overexpress Six1. Experiments knocking down Eya2 in MCF7 cells demonstrated a decrease in cyclin A1 levels and that cyclin A1 induction is reversed (FIG. 22). These data again suggest that Eya2 is the relevant cofactor of Six1 in MCF7 breast cancer cells and that Six1 needs Eya2 to stimulate cyclin A1 and thus promote cell growth. In addition, the cells were examined for activation of TGF-β signalling (FIG. 21).

If it is necessary to knock down more than one Eya member at a time due to functional redundancy (MCF7 line), each knockdown construct may be cloned into a pSuperRetro vector containing a unique selection marker to obtain multiple stably transfected cells. It should be noted that the MCF7 cells are already tagged with Zs-green to allow simple detection of metastatic lesions. Luciferase RNAi oligonucleotides or expression constructs will be used as controls in both transient and stable knockdowns. Those clones that sufficiently knock-down appropriate Eyas will be injected into the flank of nude mice, or into the #4 mammary gland for transformation and metastasis assays. In all assays, these cell lines can be compared to the control MCF12A or MCF7-CAT transfectants to determine whether the reduction of Eya in Six1-overexpressing mammary cells can return the phenotype to that of cells which do not overexpress Six1. If this occurs, it will demonstrate that Six1 is dependent on Eya(s) to mediate its proliferative, tumorigenic, and metastatic effects on breast cancer cells. As further confirmation, cells can be “rescued” for Eya expression by transfecting wild type Eyas back into the cells in which Eyas are knocked down. The transfected Eyas will be mutated in the wobble position of the codons targeted by the RNAi, and will thus encode wild type proteins that cannot be knocked down by the Eya specific siRNAs. To determine the role of the phosphatase, the inventors will attempt to “rescue” the Eya knockdown cells with phosphatase-dead Eya. If phosphatase-dead Eyas do not rescue the ability of Six1 to mediate tumorigenesis/metastasis, the inventors will have conclusive evidence that the Eya phosphatase activity is required for Six1 tumorigenicity. Determining the role of Eya's phosphatase activity is critical because small molecule inhibitors can be more easily designed to target enzymatic activities. In MCF7 cells, the two Eyas may work together to mediate proliferation and metastasis or Eya2 may be more important for proliferation whereas Eya4 will be critical for metastasis. The role of each Eya can be determined by individually knocking each Eya member down and then knocking them down together. Indeed, results in which Eya2 was knocked down in MCF7 cells demonstrate that Six1-induced increases in cyclin A1 levels and proliferation are reduced by Eya2 knockdown alone to levels observed prior to Six1 overexpression. This suggests that Eya2 plays a predominant role in Six1-induced proliferation in MCF7 cells.

The stable Eya2 knockdowns were also tested to determine whether they can reverse the ability of Six1 to induce EMT, which is heavily associated with metastasis. In these cells, it was possible to reverse some of the pro-tumorigenic phenotypes induced by Six1, including EMT. FIG. 16 demonstrates that loss of Eya2 in MCF7 cells reverses the ability of Six1 to induce a more mesenchymal phenotype in MCF7 cells, as assessed by the levels of the fibronectin protein. Similarly, the relocalization of E-cad and β-catenin is associated with EMT. Thus, a reversal of Six1 induced EMT suggests that Eya2 loss may reverse the metastatic phenotype caused by Six1.

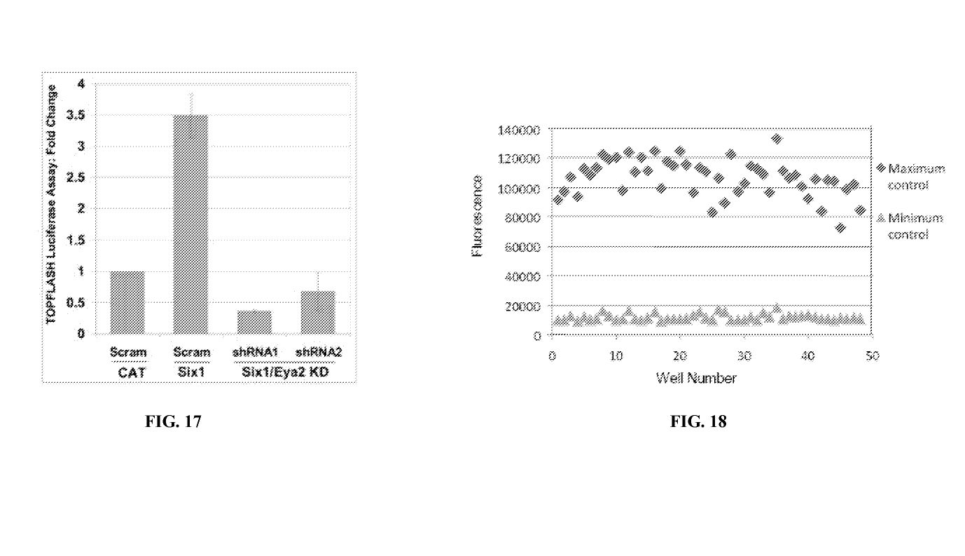

In addition, it was previously demonstrated that Six1 overexpression leads to an increase in β-catenin transcriptional activity. This is believed to be due to the fact that Six1 overexpression in MCF7 cells results in the relocalization of E-cadherin, and thus β-catenin, away from the membrane. Thus, it was examined whether Eya2 knockdown in Six1-overexpressing MCF7 cells would reverse the Six1-induced enhancement of nuclear β-catenin activity, as assessed by the pTOPFLASH reporter. Indeed, Eya2 knockdown in MCF7-Six1 cells inhibits the induction of TOPFLASH luciferase activity (FIG. 17). The data with stable knockdown of Eya2 in MCF7-Six1 cells strongly suggest that loss of Eya2 will reverse Six1 induced phenotypes and that Eya2 is an important mediator of Six1-induced tumorigenicity and metastasis, and is a viable therapeutic target.

### Example 5

### Determine the Six1-DNA Co-Crystal Structure

The inventors have expressed and purified large quantities of full-length and near full-length Six1 that binds DNA (FIGS. 4A-B). The inventors will combine purified Six1 with the DNA consensus sequence identified in Example 1 for crystallization trials. The inventors will start the crystallization trial with the minimum Six1 binding sequence and will then add one nucleotide at a time on either end of the minimum sequence to create DNA oligonucleotides for further crystallization trials. Once suitable crystals are obtained, the Molecular Replacement (MR) method can be used to determine the structure with canonical Homeodomain (HD) structures as models. However, if the HD is only a small portion of the protein crystallized, the MR method using HDs as a model may not succeed. Six1 contains four Methionines (Mets) of the total 284 amino acids, providing a good source for determining the structure using the Se-Met MAD method (Hendrickson and Ogata, 1997). If there are any unexpected complications with the Se-Met method, conventional heavy atom soaking will be performed and MIR or MAD methods can be used to determine the structure once heavy atom containing crystals are obtained.

The structure will reveal the molecular details of the Six1/DNA interaction, providing critical information needed for structure-based drug design. These details are essential for the further understanding of the molecular mechanism of Six1 and Eya's function and for structure-based drug design targeting the Six1/DNA interaction, Six1/Eya interaction, or Eya's phosphatase activity. Although protein/DNA interactions have traditionally been regarded as difficult target for drug design, there have been encouraging recent progresses (Vinson, 2005). For example, a pyrrole-imidazole polyamide inhibitor can be synthesized to mimic the specific minor groove DNA sequence recognized by a DNA-binding protein (Olenyuk et al., 2004). After screening about 20,000 chemically diverse compounds, a small molecule inhibitor was identified that inhibit the interaction between Estrogen Receptor □ and its cognate DNA sequence and blocks estrogen-dependent growth of cancer cells (Mao et al., 2008). This discovery makes the structure of Six1/DNA complex an attractive tool to rationally design such polyamides that may inhibit the Six1/DNA interaction.

In addition, the structure will provide insight into the interaction between Six1 and Eya, which can also be targeted with small molecules. Small molecules that target protein interfaces have been a subject of much recent attention (Chene et al., 2004). Successful antagonists have been developed that target important tumor-promoting protein interactions such as p53/MDM2 (Vassilev et al., 2004), Tcf/□-catenin (Lepourcelet et al., 2004), and eIF4E/4G (Moerke et al., 2007). Although phosphatase inhibitors can be fairly non-specific, there are examples of successful specific phosphatase inhibitors, such as calcineurin and PTP1B inhibitors (Boutselis et al., 2007). In addition, Eya is a unique phosphatase that uses an aspartic acid as the catalytic residue, differing from most Cys-dependent or metallophosphatases in the cell. Structural details of the Eya active site may offer us a unique opportunity to design Eya-specific phosphatase inhibitors. Since Six1 stimulates cellular proliferation via cyclin A1 in mammary carcinoma cells (Coletta et al., 2004), these compounds have the potential to inhibit Six1 mediated proliferation, tumorigenesis, and metastasis.

If the inventors have difficulty crystallizing full-length Six1 with DNA, the first alternative is to co-crystallize the near full-length Six1 with DNA. The inventors have expressed and purified the near full-length Six1 identified from the natural degradation product of Six1. The naturally occurring degradation indicates the existence of flexible regions in the protein. Removal of these regions will generate more compact proteins that are usually easier to crystallize than flexible proteins. The second alternative is to express, purify, and co-crystallize the Six Domain (SD)+HD (residues 9-183) (FIG. 3) with DNA. The C-terminal region of Six1 following the HD is highly divergent among different Six family members. It is possible that the SD+HD forms an even more compact structure than the near full-length Six1 resulting from natural degradation. The third alternative is to crystallize the HD alone with DNA. HDs usually contain three helices folded into a compact globular structure that is likely to crystallize. This structure can still provide a target for structure-based drug design to inhibit Six1/DNA interactions.

### Example 6

### Determine the Crystal Structure of Six1 In Complex with Eya

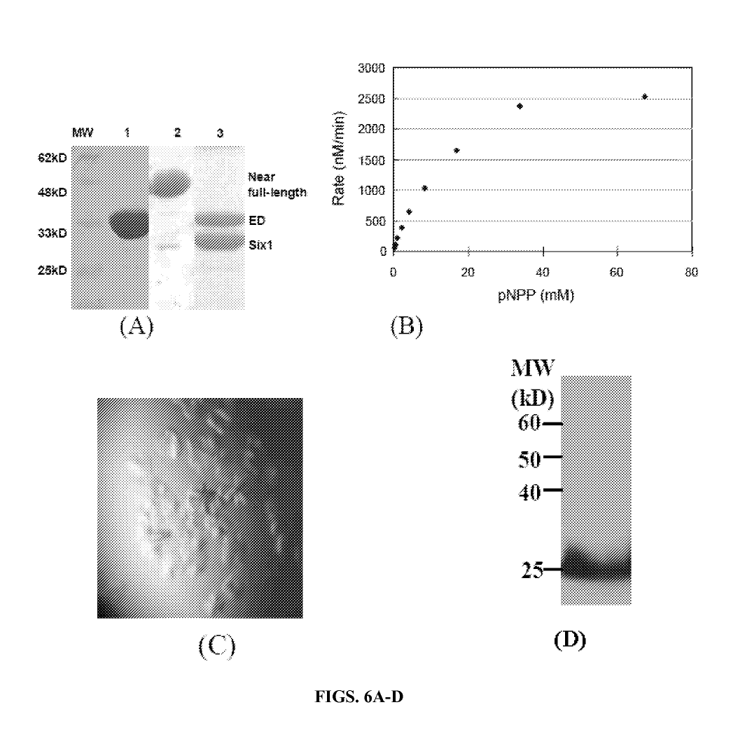

The inventors have expressed and purified large quantities of Eya2 ED from E. coli (FIG. 6A). Full-length Eya degrades into a near full-length fragment (residues 96-538) during purification and the inventors also purified large quantities of the near full-length Eya2 (FIG. 6A). The inventors will express and purify the full-length, near full-length and ED of other Eyas. The inventors demonstrated that purified Eya2 ED has phosphatase activity using the standard phosphatase substrate p-Nitrophenyl Phosphate (pNPP) (FIG. 6B). The inventors also demonstrated that Eya2 ED interacts with Six1 and forms a single complex on a gel filtration column (FIG. 6A). The inventors have obtained preliminary crystals of the Six1/Eya2 ED complex although the crystals are still too small to obtain useful diffraction data (FIG. 6C). Western blot analysis of crystals after thorough washing shows that these crystals contain Six1.

The inventors will adjust crystal growth conditions and screen for crystallization additives to improve the size of the Six1/ED crystals. Utilizing synchrotron radiation may also enable the inventors to obtain useful diffraction data from small crystals. Once a suitable crystal is obtained, the structure can be determined using MR or MIR/MAD using heavy atoms or Se-Met. Structure of the Eya2/Six1 complex will provide insights into how the ED interacts with Six1, the molecular mechanism of Eya's phosphatase activity and structural targets for inhibiting the Six1/Eya interaction or Eya's phosphatase activity.

Once these structures are available, the inventors will perform computer based virtual library screening to identify small molecule compounds that will inhibit the Six1/DNA interaction, the Six1/Eya interaction, or Eya's enzymatic activity. Once the inventors have reached this stage, the inventors will synthesize lead compounds identified from virtual library screening and characterize their dose response, selectivity, and mechanism of action. In the future, the inventors will also identify inhibitors targeting the Six1/DNA interaction, Six1/Eya interaction, and Eya phosphatase activity through high throughput screening approaches to complement structure-based drug design.

In addition, the inventors will also co-crystallize other Eyas (full-length, near full-length, or ED) with Six1 to increase the chance of obtaining well diffracting crystals. Homologous proteins are often used to overcome crystallization difficulties. Although these proteins may only differ by a few amino acids, their crystallization properties can be dramatically different. The EDs of all Eyas are highly conserved and structure of any Eya/Six1 complex will provide insights in to the Six1/Eya interaction, Eya's phosphatase activity and provide targets for rational drug design.

### Example 7

### Identification of Eya Inhibitors Through High Throughput Screening (HTS)

The initial effort will focus on developing inhibitors targeting Eya2 due to the large quantities of pure Eya2 ED the inventors already obtained and Eya2's involvement in ovarian and pancreatic cancers. The ED (which harbors the phosphatase activity and is responsible for interacting with Six1) of all Eya proteins are highly conserved (sequence identity between 65 and 89%). The inventors will evaluate the effects of Eya2 inhibitors against other Eya and expand the HTS to include other Eya proteins. The inventors will initially use the phosphatase assay the inventors developed with pNPP as substrate for HTS since the assay can be easily adapted to the HTS format. The inventors will perform HTS using the NCI diversity set of 1990 compounds to test the ability of these compounds for inhibiting Eya2 ED's phosphatase activity Inhibitors identified will be further evaluated for their ability to inhibit Eya's phosphatase activity using peptide substrate.

Dose response curves are generated and IC50 values are obtained for promising hits and perform kinetic analyses of ED in the presence of inhibitor. These analyses will provide clues whether the small molecule compound is competitive or non-competitive inhibitors. Counter screen is performed to examine the specificity of these primary hits. Specifically, it will be evaluated whether these primary hits inhibit a number of other phosphatases, including SHP-1, SHP-2, CDC25A, CDC25B, CDC25C, MKP3 phosphatase, KAP, PRL-3. To understand the mechanism of action of these compounds, the Six1/ED crystals are soaked with the inhibitor or co-crystallize ED with the inhibitor. If crystallization is difficult, NMR chemical shift perturbation can be used to identify inhibitor binding sites on ED. The crystal structure of ED (or Six1/ED) with the inhibitor is particularly useful in further optimizing the initial lead. The inventors have demonstrated that the purified ED has phosphatase activity using pNPP as a substrate (FIG. 6B). Km and kcat of the reaction is 16 mM and 11/min, which falls in between the kinetic values reported by two groups for mouse Eya3 ED (Rayapureddi et al., 2003; Tootle et al., 2003).

Since Eya proteins can utilize their intrinsic phosphatase activity to switch the Six1 transcriptional complex from a repressor to an activator complex, it is highly possible that Eya's phosphatase activity is required for Six1-mediated breast tumorigenesis and metastasis Inhibitors targeting the Eya active site can possibly cause conformational changes and indirectly affect the Six1/Eya interaction and inhibit Six1 's transcriptional activity. In addition, small molecules that effectively and specifically inhibit the Eya phosphatase can be valuable chemical probes to understand the function of Eya's phosphatase activity in the cell.

A high throughput phosphatase assay has been developed using the small molecule substrate 3-O-methyl-fluorescein phosphate (OMFP), which is converted to a fluorescent product OMF upon dephosphorylation. The enzymatic condition (buffer pH, salt concentration, Mg++ concentration) has been optimized and the Km for Eya2 ED (463±104 μM) was determined. The inventors chose 100 μM OMFP (<Km), 50 nM Eya2 ED, and an one-hour incubation time as initial conditions for the HTS assay, which produces a linear enzymatic response within the incubation time and significant signal to background ratio (>=5). The Z-factor of the assay was evaluated using DMSO-only as the maximum control and no Mg++ (which completely abolishes phosphatase activity) as the minimum control. The Z-factor for the assay is 0.52, indicating a valid HTS assay (FIG. 18).

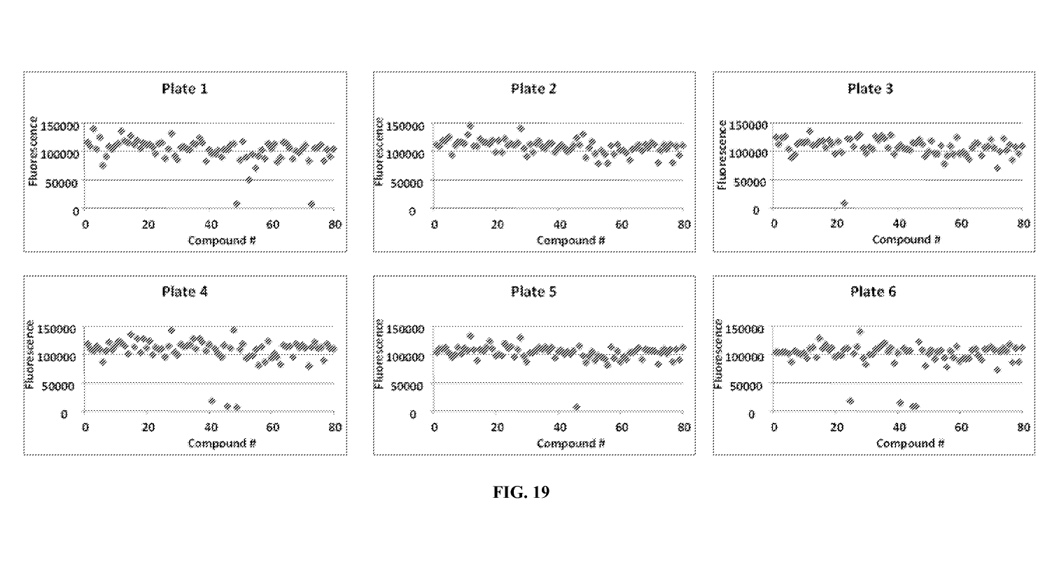

The inventors screened 480 compounds from six plates in the NCI diversity set (1,990 compounds total) at 10 μM concentrations (FIG. 19). Plate 2 does not have any obvious hit but all other plates have 1-3 hits that almost completely abolished the fluorescence signals. Thus, HTS can be successfully developed and carried out. These screens will be repeated and completed for an entire NCI set. The compounds that completely abolish the fluorescence signals are selected for further in vitro and in vivo characterizations.

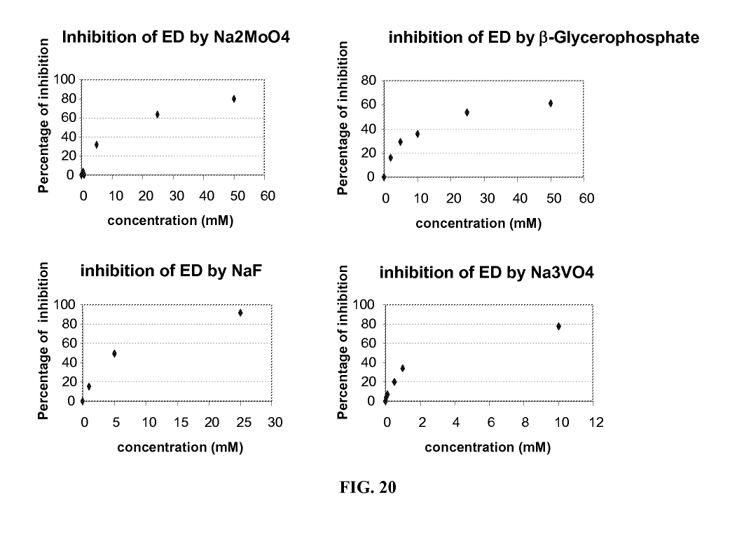

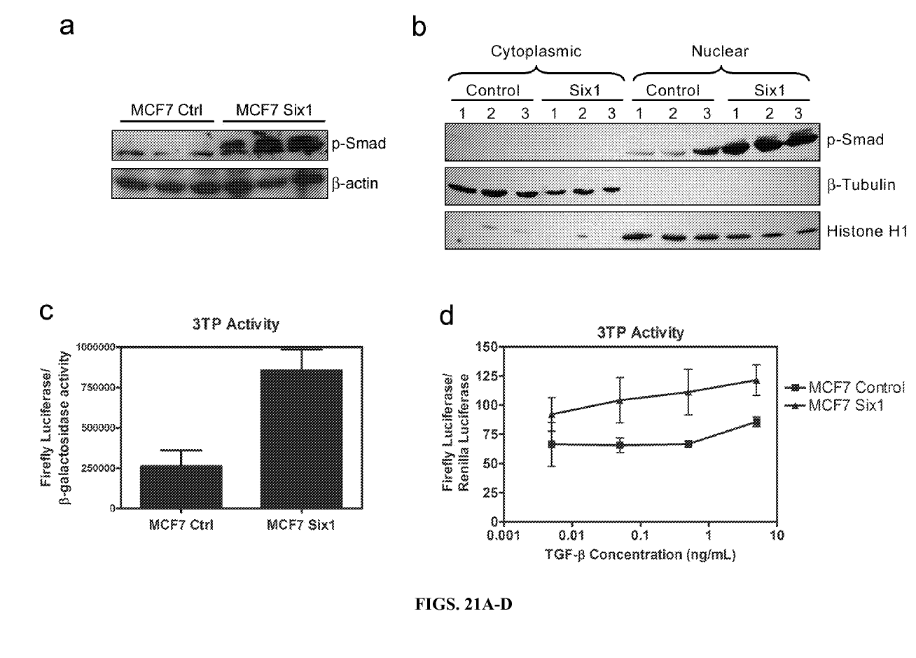

Nine known phosphatase inhibitors were tested for the ability to inhibit the phosphatase activity of human Eya2 Eya Domain (ED). It was found that five of these inhibitors have no effect on ED at the concentrations that typically completely inhibit their cognate phosphatases. These inhibitors are: Okadaic acid (inhibitor of Ser/Thr phosphatase PP2A), L-phenylalanine (intestinal alkaline phosphatase inhibitor), cyclosporine A (binds cyclophilin and inhibits calcineurin), (1, 10)-phenanthroline, phenylarsine oxide (protein tyrosine phosphatase inhibitor). The other four inhibitors demonstrate some inhibition of ED (FIG. 20). The IC50 of these inhibitors are: Na2MoO4: 11.3±1.6 mM, β-glycerophosphate: 8.2±2.1 mM, NaF: 6.6±0.3 mM (typically considered as a broad spectrum Ser/Thr phosphatase inhibitor), Na3VO4 (broad spectrum protein tyrosine phosphatase inhibitor): 1.8±0.2 mM. The fact that not all known phosphatase inhibitors inhibit ED's phosphatase activity and the ones that do inhibit have very high IC50s suggest that the active sites of Eya2 ED are significantly different from these known phosphatases. Thus, it may be possible to identify inhibitors that are specific to Eya2 ED and do not significant effect on other phosphatases.

### Example 8

### Assay for HTS of Small Molecule Compounds that Inhibit the Six1/DNA Interaction

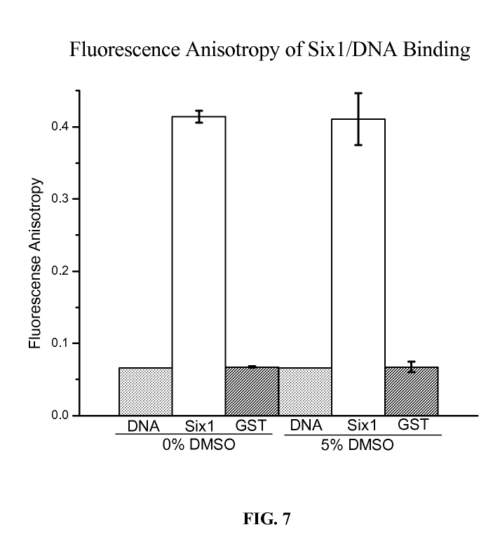

A fluorescence polarization assay, which is the most commonly used assay for HTS of compounds targeting protein/DNA interactions, is developed. A similar assay has been successfully used for HTS to identify small molecules that disrupt the DNA binding of B-ZIP transcription factors (Rishi et al., 2005). Preliminary data using a single cuvette (FIG. 4D) demonstrated that the binding of the 28 kD Six1 near full-length protein to the DNA (10 kD) significantly increased the fluorescence polarization of the DNA (>4-fold). Fluorescence anisotropy experiments demonstrate that Six1+ED binds to the myogenin MEF3 sites, whereas the non-specific GST protein does not. (FIG. 7) In the next step, the experiment is adapted to a 96-well format to evaluate the Z-factor for the assay. Z-factor is defined as 1-3×SSR/R(SSR is the summation of the standard deviation of positive controls and negative controls; R is the mean of the positive controls minus the mean of negative controls). Z-factor provides a quantitative assessment of the quality of a HTS assay, reflecting both the assay signal dynamic range and the data variation associated with the signal measurements. In the assay, the positive control is unlabeled DNA which should compete with fluorescein-labeled DNA and mimic the effect of a compound that inhibits the Six1/DNA interaction. The negative control is DMSO alone which should not have significant effect on the polarization. Positive control and negative control is measured multiple times to evaluate Z-factor. Experimental conditions are fine tuned (such as quantity of fluorescein-labeled DNA, quantity of protein, and buffer condition) to reach an ideal Z-factor (0.5<z≦1).

The HTS assay is applied to the freely available NCI diversity set of 1990 compounds. These compounds were selected from 140,000 compounds based on the criteria of chemical diversity and has been successfully used to identify lead compounds inhibiting the b-ZIP transcription factor and DNA interaction (Rishi et al., 2005). There are currently no known libraries enriched in protein/DNA inhibitors and a chemically diverse library is the best starting point to identify Six1/DNA inhibitors. Aliquots of small molecule compounds are added to the 96-well plate and incubated with fluorescein-labeled dsDNA and Six1 near full-length protein. Fluorescence polarization is measured and compared to that of the fluorescein-labeled dsDNA, fluorescein-labeled dsDNA+Six1 near full-length, and fluorescein-labeled dsDNA+Six1 near full-length+unlabeled dsDNA. The screen is repeated independently for three times to identify compounds that consistently result in significant decrease (>50%) of fluorescence polarization.

Ten compounds that result in the most inhibition are selected for follow-up studies. A secondary assay such as EMSA is used first to confirm the effect of these compounds on Six1/DNA interaction. The dose response curve (IC50) of promising compounds is determined. The specificity of these compounds is examined by whether they inhibit the interaction between other homeobox proteins and their DNA targets. The homeoproteins belong to several other homeobox families including the Hox (e.g., HoxB1), Pax (e.g., Pax5), Msx (e.g., Msx-1), and Lim (e.g., Lhx9) families. HDs can be easily expressed and purified as recombinant proteins in E. coli, demonstrated by the fact that members of all above homeobox families have been subjected to crystallographic or NMR studies (Piper et al., 1999; Hovde et al., 2001; Garvie et al., 2001). Since the Six-type HD is divergent from “classical” HDs and binds a somewhat different DNA consensus sequence (Christensen et al., 2008), an inhibitor that specifically targets the Six1/DNA interaction should be identifiable while not affecting the interaction between other HDs and DNA. In addition, it will be determined whether the inhibitors target the interaction of other Six family members with DNA. The inhibitors may target the interaction of other Six homeoproteins with DNA, but do not anticipate this to be problematic as like Six1, these other Six family members are primarily expressed during embryogenesis and lost in adult tissues (Christensen et al., 2008). Furthermore, if the other Six family members were expressed out of context in a tumorigenic setting, their inhibition would likely also inhibit tumor proliferation and survival, as most Six family members tested to date are pro-proliferative and pro-survival. The mechanism of action of promising compounds is analyzed using biochemical/structural approaches.

Whether a compound inhibits the Six1/DNA interaction by binding to DNA or Six1+ED will be determined using the following methods. EtBr displacement assay (Boger et al., 2001) is performed to determine whether a compound inhibits the Six1/DNA interaction by binding to DNA. This assay is based on the fact that EtBr forms a fluorescent complex when bound to DNA and a DNA-binding compound displaces pre-bound EtBr, resulting in decreased fluorescence. For compounds that are shown to bind DNA, the DNA sequence is varied in the EtBr displacement assay to evaluate whether these compounds bind specific DNA sequences. By altering the DNA sequence, the inventors will be able to determine if a compound binds to DNA with or without sequence specificity. For compounds that do not bind DNA, it is evaluated whether they interact with Six1 using biophysical approaches such as Surface Plasmon Resonance (SPR). Recent advances in SPR has enabled it to routinely detect the binding of small molecules (<500 Da) (Myszka and Rich, 2000). The crystal structure of Six1 with the Six1-binding compounds is determined or map the compound binding sites on Six1 using NMR chemical shift mapping. An ideal compound(s) will have a reasonable IC50 (in the □M range or lower) and inhibit Six1/DNA interaction by specifically interacting with DNA or Six1 itself. These compounds are valuable lead compounds for developing high affinity and high specificity Six1/DNA inhibitors. The effect of promising compounds on Six1's in vivo activity is evaluated using cellular assays such as Luciferase, transformation, or proliferation assays. Further HTS is performed at the Broad Institute, which has 250,000 compounds freely available and include many commercial compound libraries, NCI collections, known bioactives collections, natural product collections, and diversity oriented synthesis collections.

The feasibility of developing a fluorescence anisotropy-based HTS assay targeting the Six1/DNA interaction was evaluated. Fluorescence anisotropy is the most commonly used assay for HTS of compounds targeting protein/DNA interactions. In this assay, fluorescein labeled DNA tumbles fast by itself and has low fluorescence anisotropy values. Upon binding to proteins, the protein/DNA complex tumbles much slower and demonstrates an increased fluorescence anisotropy value. A similar assay has been successfully used to identify small molecules that disrupt the interaction between B-ZIP transcription factors and DNA (Rishi et al., 2005). The Six1+ED complex was used instead of Six1 alone for the fluorescence anisotropy experiments since ED significantly increase Six1's DNA binding affinity (FIG. 7) and Six1+ED is a more realistic representation of the situation in the cell where ED is required for Six1's transcriptional activity. Furthermore, the larger Six1+ED complex will also increase the fluorescence polarization signal compared to Six1 alone. In preliminary studies, fluorescence anisotropy values of 5′-fluorescein labeled dsDNA (16 nt containing the MEF3 DNA binding site) and fluorescein-labeled MEF3 DNA+Six1/ED complex (or a non-specific protein, GST, that should not bind the DNA), were measured using a single cuvette, at an excitation wavelength of 485 nm, and emission wavelength of 535 nm. The addition of Six1/ED leads to a three-fold increase in fluorescence anisotropy over labeled DNA alone, while DMSO (in which screening compounds are dissolved) has no effect on fluorescence anisotropy (FIG. 25).

### Example 9

### Assay for HTS of Small Molecules Compounds that Inhibit the Six1/Eya Interaction

An ELISA assay is developed for HTS targeting the Six1/Eya interaction. Eya proteins interact with Six1 through its Eya domain (Jemc and Rebay, 2007. Since ED of all Eya proteins are highly conserved (83-89% sequence identity), Six1/Eya2 ED serves as a good model system for HTS targeting the Six1/Eya interaction in general. ED from other Eyas is expressed and purified. Compounds effective in inhibiting the Six1 and Eya2 ED interaction are be evaluated for their effect on the Six1 and other ED interactions. Although Eya2 seems to be playing a more dominant role in various cancers than other Eyas, it is possible that other Eyas may compensate once the Six1/Eya2 ED interaction is inhibited. Therefore, an ideal compound should have a broad spectrum activity that inhibits the interaction between Six1 and all Eyas, which is possible since the EDs of different Eyas have high sequence conservation.

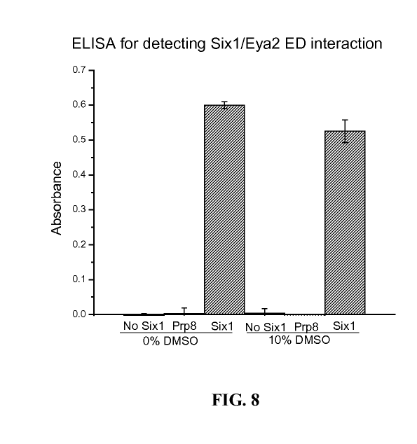

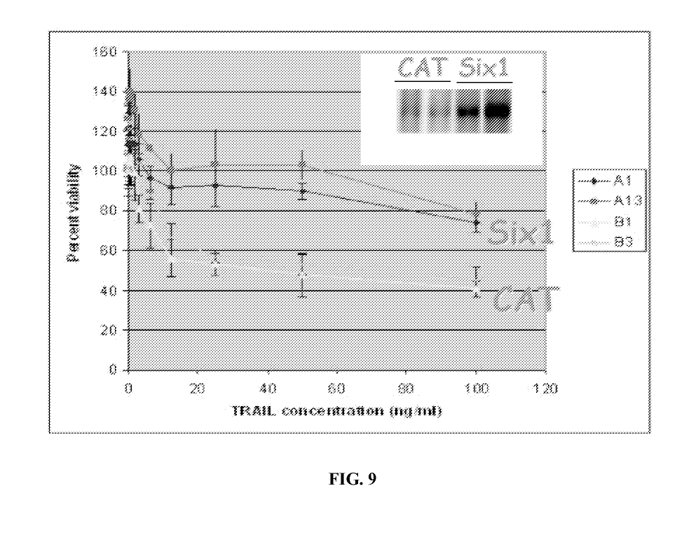

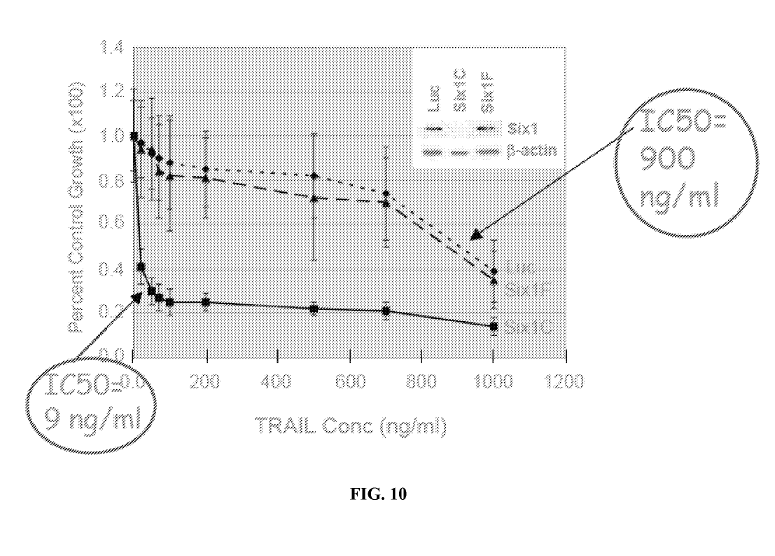

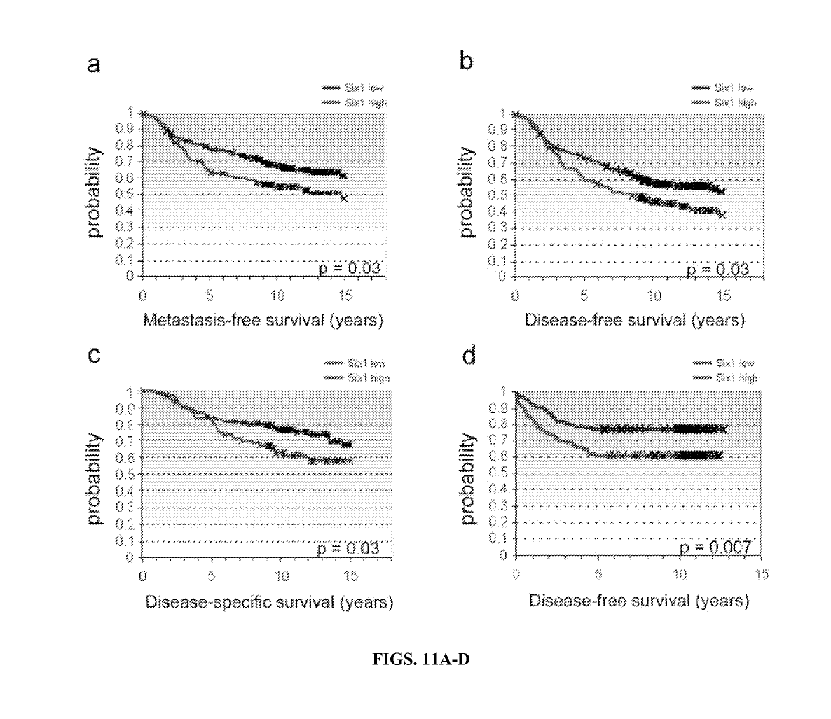

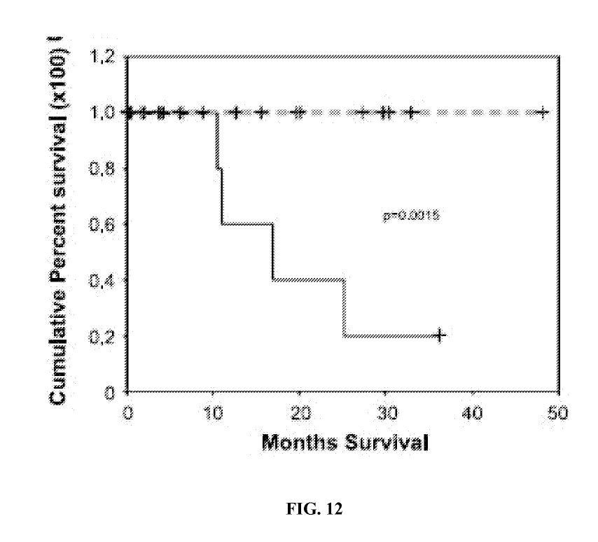

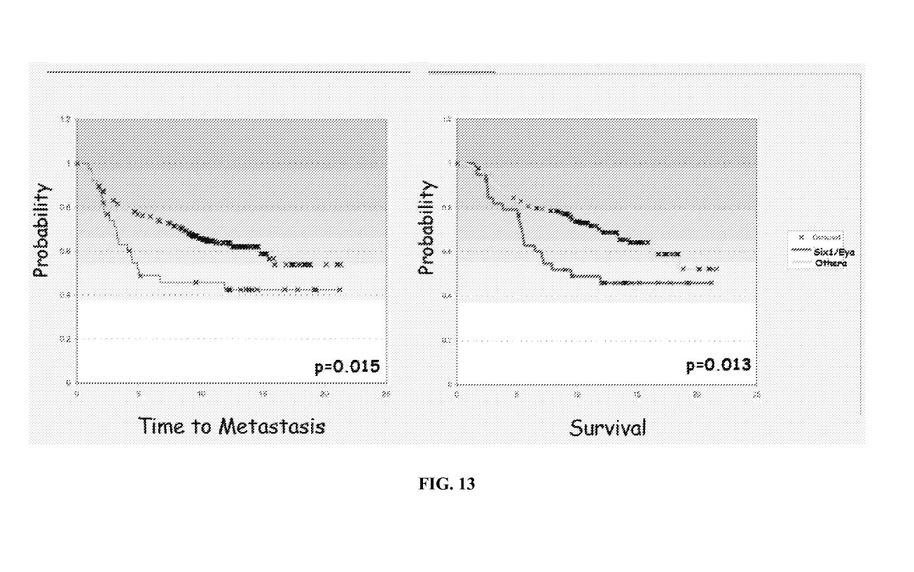

To develop ELISA, the ELISA plate is coated with Eya2 ED and incubated with Six1. This is followed by incubation with anti-Six1 antibody and subsequent incubation with secondary antibody coupled with Horseradish Peroxidase (HRP). When Six1 binds Eya2 ED, the secondary antibody is captured on the ELISA plate, which can be monitored using a spectrometer after reacting with ABTS (an HRP substrate) (FIG. 8). The ELISA assay is used to screen the NCI diversity set of 1990 compounds. Since there is currently no library enriched in protein/protein interaction inhibitors, the chemically diverse library provides us the best opportunity for identifying Six1/ED interaction inhibitors. Similar to the HTS targeting Six1/DNA interactions, initial hits are further validated using secondary assays such as SPR. The IC50 is evaluated, including whether these compounds specifically inhibit the Six1/ED interaction. The mechanism of action of these compounds is determined using biophysical and structural approaches.

Alternatively, the ELISA plate may be coated with Six1 and detecting with an anti-ED antibody. This alternative experimental set up should increase the chance of developing a successful ELISA assay. A FRET or fluorescence polarization assay may be developed to monitor the Six1/ED interaction if ELISA turns out to be unsuccessful. Six1 and Eya2 ED are similar in size which may not generate significant fluorescence polarization changes. However, using a GST or MBP-fused ED will significantly increase the size of ED and may resolve this problem. Still another method may be to use an AlphaScreen and time-resolved fluorescence will likely be valuable alternatives to target Six1/Eya interactions. An EnVision plate reader (Perkin Elmer) can perform AlphaScreens and time-resolved fluorescence, which can be used to develop these assays.

Although ELISA may be usable for a low or medium throughput screening, it is unsuitable for large scale HTS due to its multiple wash steps. A fluorescence polarization assay for HTS will be developed. The inventors will covalently attach fluorescein on Six1 using NHS-fluorescein which reacts with primary amines of Lys on protein surface. The addition of the ED to Six1 will likely cause an increase of fluorescence. If the fluorescence increase is not obvious, the inventors will use GST-ED fusion proteins. Since GST is a dimer, GST-ED will be roughly 100 kD. The large size of GST-ED fusion protein should have a much better chance than ED alone to significantly increase fluorescence polarization upon binding to Six1. If for some reason the labeling efficiency for Six1 is low, the inventors will fluorescein-label ED and add Six1 for fluorescence polarization experiments. Once the Six1/Eya2 ED structure is available, a peptide in Six1 or Eya may be labeled with fluorescein if the inventors can identify a peptide that is critical for binding. A similar strategy has been successfully used for HTS of small molecules targeting the eIF4E/eIF4G protein/protein interactions (Moerke et al., 2007) using a fluorescein-labeled eIF4G peptide. HTS using fluorescence polarization identified a small molecule that binds to eIF4E, disrupt eIF4E/4G interaction, inhibits cellular expression of multiple oncogenic proteins, and exhibit activity against different cancer cell lines but not untransformed cells, demonstrating the potential of an inhibitor targeting protein/protein interactions (Moerke et al., 2007).

The fluorescence polarization assay will be adapted to a HTS format. Assay conditions will be optimized to achieve ideal Z-factor (between 0.5 and 1), similar to what was done for the fluorescence polarization assay described for Six1/DNA interactions. In this case, the maximum control will be DMSO only. If Six1 is fluorescein-labeled, the minimum control will be the addition of large amount of unlabeled Six1 to compete for binding to ED. Once a HTS assay is established, the NCI diversity set of 1990 compounds will be screened. Screening of the small library will provide an opportunity to fine tune the HTS assays. The HTS will then be performed on a much larger scale (300,000 compounds) through the NIH MLPCN.

Similar to the HTS targeting Six1/DNA interactions, initial hits will be further validated using secondary assays such as ELISA or SPR. In SPR, the inventors can immobilize Six1 (or ED) on a SPR chip and flow ED (or Six1) through the sample chamber to monitor Six1/ED interaction and generate on-rate, off-rate, and Kd values. Inclusion of inhibitors in the SPR buffer should reduce Six1/ED interaction. Dose responses may be measured using fluorescence polarization or ELISA to determine the IC50 of promising compounds.

Specificity is not a particular concern in this case. Compounds effective in inhibiting the Six1 and Eya2 ED interaction will eventually also be evaluated for their effect on the Six1 and other ED interactions both in vitro and in living cells.

NMR chemical shift mapping will be an ideal method to determine whether the inhibitor binds to Six1 or ED to inhibit their interaction. ¹⁵N-labeled Six1 or ED from E. coli is prepared using minimal media and ¹⁵NH₄Cl. TROSY-HSQC spectrum of ¹⁵N-labeled Six1 or ED by themselves and in the presence of inhibitors is collected. If an inhibitor binds Six1 or ED, the TROSY-HSQC spectrum in the presence of inhibitor will demonstrate significant chemical shift changes. The inventors can also assign chemical shifts in Six1 or ED's HSQC spectrum to specific residues in these proteins and use this information to identify residues that interact with the inhibitor. The crystal structure of Six1 or ED with promising inhibitors is determined. Since these inhibitors are expected to disrupt the Six1/ED interaction, soaking the Six1/ED crystals with inhibitors will likely be ineffective, so it is necessary to co-crystallize Six1 or ED with these inhibitors. Once suitable crystals are obtained, the Molecular Replacement method utilizing the Six1/ED structure can be applied to determine the inhibitor-containing crystals. The detailed interaction between an inhibitor and Six1 or ED is invaluable in optimizing these inhibitors.

### Example 10

### Assessment of Compounds in Cell Culture

Compounds that have high potency and specificity will be further tested in cell culture models for their ability to inhibit Six1-mediated transcriptional activation, and to inhibit cell growth and survival. The inventors will first test whether these compounds can inhibit Six1-mediated transcription from the MEF3 reporter gene. To accomplish this task, the inventors will use the pGL3-MEF3-luciferase (pGL3-MEF3-luc) reporter construct first described by Manning and colleagues (Fan et al., 2000). This reporter construct contains 6 contiguous MEF3 DNA binding sites upstream of a TATA box, and Six1, in complex with Eya proteins, is known to strongly activate this promoter resulting in luciferase expression. The reporter will be transfected into MCF7 cells into which both Six1 and Eya2 will be co-transfected to allow for maximal stimulation of the reporter. Lead compounds (or vehicle only) will be added to the cells at varying concentrations, to determine whether they can inhibit Six1-mediated transcriptional activation in a dose-dependent manner. The IC50 will be calculated for all compounds tested.

Once the inventors determine which lead compounds are most effective at inhibiting Six1-mediated transcription, the inventors will assess their ability to inhibit cell proliferation and survival. To accomplish this task, the inventors will treat both normal mammary epithelial cells lines expressing little to no Six1 (MCF10A, MCF12A, 16N) (Reichenberger et al., 2005, and data not shown) and mammary carcinoma cells expressing intermediate to high Six1 endogenously (MCF7, T47D, ZR-75-1, 21NT, 21PT1, 21MT1, 21MT2) (Reichenberger et al., 2005), as well as MCF12A and MCF7 control and Six1 overexpressing lines with varying doses of the lead compounds. The inventors will then determine, using Alamar blue staining, cell counts, and bromodeoxyuridine (BrdU) incorporation assays, whether inhibition of Six1-mediated transcription in cancer cells, will inhibit cell growth. This analysis will allow us to determine the IC₅₀ is for each compound. The inventors have previously shown that Six1-mediated proliferation is dependent on its ability to transcriptionally activate cyclin A1. Thus, to ascertain whether inhibition of cell proliferation by lead compounds is dependent on the ability of these compounds to inhibit Six1-mediated transcription, the inventors will first examine cyclin A1 levels in the cell in response to drug treatment, and the inventors will then determine whether the inventors can rescue the inhibition of cell growth by introducing pcDNA3.1-cyclin A1, whose promoter cannot be controlled by Six1 (Coletta et al., 2004).

As Six1 enhances cell survival (Behbakht et al., 2007; Yu et al., 2006), particularly via inducing resistance to TRAIL-mediated apoptosis (Behbakht et al., 2007), lead compounds may increase basal levels of apoptosis, as measured by annexin V staining followed by flow cytometry, and the compounds may increase sensitivity of cells expressing Six1 to TRAIL-mediated apoptosis (Behbakht et al., 2007). In addition, lead compounds may inhibit the ability of Six1 to induce TGF-β signaling (in both MCF12A and MCF7 cells), and may be able to reverse the EMT induced by Six1 in both the MCF12A and MCF7 cells.

### Example 11

### Assessment of Compounds in Animal Models

To carry out the proof of principle in vivo experiments, the inventors will focus on the best lead compound that showed significant and specific inhibition of Six1 activity both in vitro and in cell culture, and did not show significant activity against normal mammary epithelial cells that do not express Six1. The inventors will first test this compound for the maximum tolerated dose (MTD) in vivo. This will be done by administering the compound to nude mice or NOD/SCID mice (the model animals to be used) either via oral gavage, intraperitoneal (i.p.) injection, or intravenous (i.v.) injection, based on its chemical composition and expected properties. In addition, the frequency of administration and the doses to be given will be based on known properties of the compound (or related compounds), and on the doses used to effectively inhibit Six1 activity in vitro. Once the MTD is established, the inventors will perform the experiments outlined below to determine whether inhibition of the Six1 transcriptional complex can: 1) inhibit tumor formation, 2) inhibit tumor growth, and 3) inhibit metastasis.

In the first set of experiments, the inventors will determine whether the lead compound can inhibit the ability of Six1 to induce tumorigenesis. To perform this experiment, the inventors will inject 2×10⁶ cells of the three MCF12A-Six1 clonal lines and the three MCF12A-Control (ctrl, CAT) lines into the left and right #4 mammary glands, respectively, of nude mice that have been supplemented with estrogen (Coletta et al., 2008). As early as three days after injection (before obvious tumors are formed), mice from the MCF12A-Six1 lines will be treated with various concentrations (at least 3 different concentrations) of the lead compound (up to but not exceeding the MTD), or a vehicle control. The mice will continue treatment (at a frequency of dosing determined as outlined above) for up to two months, allowing time for MCF12A-Six1 vehicle tumors to get to a significant size, not exceeding 2000 mm³. During the dosing period, mice will be sacrificed if they have lost 15% of their body weight or are moribund as judged by the veterinary staff. Tumor size will be measured with calipers twice weekly, and volume reported in mm³, calculated by using the formula volume=0.5×length×width². Significance in differences in tumor size in drug versus vehicle treated will be calculated using the Kruskall-Wallis one-way analysis of variance test. At the end of the study (2 months), all mice will be sacrificed via cardiac exsanguination under isofluorane anesthesia to allow the analysis of plasma concentrations of the drug using mass spectrometry (Kelly et al., 2002). In addition, all tumors will be removed and weighed. This is designed to determine whether the small molecule lead compounds can inhibit the ability of Six1 to induce tumor formation.

In a second set of experiments, the inventors will determine whether administration of lead compounds can cause already formed tumors to regress or to grow less rapidly. the inventors will perform this second experiment because it is this scenario that will arise in the clinic. The analysis of whether lead compounds can inhibit the growth of already formed tumors will be performed in the model of subcutaneous growth of MCF7-Six1 cells in nude mice. In the MCF7-Six1 subcutaneous tumor growth model, Six1 overexpression in MCF7 mammary carcinoma cells leads to increased tumor burden when the cells are grown in the flank of nude mice (Coletta et al., 2004). Thus, nude mice can be injected subcutaneously with MCF7-CAT cells (which do form tumors, but the tumors are not as large is MCF7-Six1 tumors), or with the MCF7-Six1 cells. The inclusion of the MCF7-CAT control tumors in this experiment will allow us to determine whether the lead compounds also cause regression of tumors that are not engineered to overexpress Six1 (these MCF7-CAT cells do express low levels of Six1 endogenously). Once the tumors reach a volume of 250 mm³, animals will be treated with varying doses of the lead compound (at least 3 different doses) or vehicle control. As outlined above, tumor size will be measured with calipers twice weekly, and volume reported in mm³, calculated by using the formula volume=0.5×length×width². Significance in differences in tumor size in drug versus vehicle treated will be calculated using the Kruskall-Wallis one-way analysis of variance test. At the end of the study (2 months or when tumors reach 2000 mm³, whichever comes first), all mice will be sacrificed as outlined above, via cardiac exsanguination under isofluorane anesthesia to allow the analysis of plasma concentrations of the drug using mass spectrometry (Kelly et al., 2002). All tumors will be removed and weighed at the end of the study, and after weighing, half the tumor will be stored in RNAlater (Qiagen) at −80° C. for subsequent analysis of RNA (to allow assessment of the expression of Six1 target genes such as cyclin A1), and the other half will be fixed for subsequent analysis of proliferative (mitotic figures and/or Ki67 staining) and apoptotic (activated caspase 3 and/or TUNEL) markers. For the immunohistochemical analysis, a reproducible semi-quantitative analysis will be performed as described (Vigneswaran et al., 2000). After examining the entire section, five representative areas will be evaluated under high power. In each field, cells will be classified with respect to their staining intensity (from 0 to 4), and the percentage of positive cells estimated. A numerical value for each field will then be calculated by multiplying the proportion of positive cells by the numerical value of that intensity. For statistical analysis the mean of each sample will be used. These experiments will be performed to determine whether Six1 inactivation via small molecule inhibition leads to a decrease in cellular proliferation and increase in apoptosis in tumors already formed at the time of lead compound administration.

The third set of experiments will be designed to determine whether the lead compound can inhibit the ability of Six1 to induce metastatic disease. In this model, the inventors will use the Zs-Green tagged MCF7-Ctrl and MCF7-Six1 overexpressing cells, and inject them orthotopically into the #4 mammary gland of NOD/SCID mice supplemented with estrogen as previously described (Micalizzi et al., submitted and preliminary data). The inventors will use the NOD/SCID model, rather than the nude mouse model, as Six1 induces metastasis in 75% of NOD/SCID mice, as compared to 40% of nude mice, likely due to the fact that NOD/SCIDs are more severely immunocompromised than nude mice. Thus, using NOD/SCID mice will allow us to reduce the number of animals needed to reach statistical significance in this study (see vertebrate animals section for mouse number calculations). The inventors will allow the tumors to establish to 250 mm³, at which time the inventors will administer varying doses of lead compounds (at least 3 different doses per compound) via either oral gavage, i.p. injection, or i.v. injection at a pre-determined frequency based on the drugs characteristics, the in vitro experiments, and the MTD experiments. The inventors will then trace the tumors, and any arising metastases, over a period of time using the Illumatool Bright Light System LT-9900. Based on previous experience, the inventors expect that fluorescent tumors will be observed in the live mice after 3-4 weeks. All mice will be sacrificed when the tumors reach 2 cm³, to ensure that size of the tumors does not influence the number of metastases, since tumor size is significantly correlated with breast cancer metastasis (Minn et al., 2007). As outlined for other animal studies, lead compound levels in the plasma will be assessed by mass spectrometry at time of sacrifice. Primary tumors will be isolated and stored for use both in RNA analysis and for histologic and immunohistochemical analysis. In addition to examining the primary tumors for markers of proliferation and apoptosis, markers of activated TGF-□ signalling (nuclear Smad-3 and phospho Smad2/3) and for EMT (E-cadherin and □-catenin) will be examined in these tumors (see preliminary studies for methods) to determine whether inhibition of TGF-β signalling and/or EMT correlates with decreased metastases. All immunohistochemical analyses will be performed as outlined above. Logistic regression with metastasis as the dependent variable and Six1 and drug dose as independent variables will be used to evaluate the difference in metastasis rates for control and Six1 xenografts either treated with drug or vehicle control.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,554,101
U.S. Pat. No. 4,683,195
U.S. Pat. No. 4,683,202
U.S. Pat. No. 4,683,202
U.S. Pat. No. 4,684,611
U.S. Pat. No. 4,800,159
U.S. Pat. No. 4,883,750
U.S. Pat. No. 4,952,500
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,302,523
U.S. Pat. No. 5,322,783
U.S. Pat. No. 5,324,633
U.S. Pat. No. 5,384,253
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,464,765
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,525,464
U.S. Pat. No. 5,527,681
U.S. Pat. No. 5,529,756
U.S. Pat. No. 5,532,128
U.S. Pat. No. 5,538,877
U.S. Pat. No. 5,538,880
U.S. Pat. No. 5,545,531
U.S. Pat. No. 5,547,839
U.S. Pat. No. 5,550,318
U.S. Pat. No. 5,554,501
U.S. Pat. No. 5,556,752
U.S. Pat. No. 5,561,071
U.S. Pat. No. 5,563,055
U.S. Pat. No. 5,563,055
U.S. Pat. No. 5,571,639
U.S. Pat. No. 5,580,726
U.S. Pat. No. 5,580,732
U.S. Pat. No. 5,580,859
U.S. Pat. No. 5,589,466
U.S. Pat. No. 5,591,616
U.S. Pat. No. 5,593,839
U.S. Pat. No. 5,599,672
U.S. Pat. No. 5,599,695
U.S. Pat. No. 5,610,042
U.S. Pat. No. 5,610,287
U.S. Pat. No. 5,624,711
U.S. Pat. No. 5,631,134
U.S. Pat. No. 5,639,603
U.S. Pat. No. 5,654,413
U.S. Pat. No. 5,656,610
U.S. Pat. No. 5,658,734
U.S. Pat. No. 5,661,028
U.S. Pat. No. 5,665,547
U.S. Pat. No. 5,667,972
U.S. Pat. No. 5,693,762
U.S. Pat. No. 5,695,940
U.S. Pat. No. 5,700,637
U.S. Pat. No. 5,702,932
U.S. Pat. No. 5,736,524
U.S. Pat. No. 5,739,169
U.S. Pat. No. 5,744,305
U.S. Pat. No. 5,780,448
U.S. Pat. No. 5,789,215
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,155
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,624
U.S. Pat. No. 6,004,755
U.S. Pat. No. 6,020,192
U.S. Pat. No. 6,054,297
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Patent Pubn. 2006/0078903
U.S. Patent Pubn. 2008/0009439
Abbondanzo et al., Breast Cancer Res. Treat., 16:182(151), 1990.
Abdelhak et al., Hum. Mol. Genet., 6(13):2247-2255, 1997.
Allred et al., Arch. Surg., 125(1):107-113, 1990.
Ando et al., Febs J., 272(12):3026-3041, 2005.
Arap et al., Cancer Res., 55(6):1351-1354, 1995.
Austin-Ward and Villaseca, Revista Medica de Chile, 126(7):838-845, 1998.
Ausubel et al., In: Current Protocols in Molecular Biology, John, Wiley & Sons, Inc, NY, 1994; 1996.
Baichwal and Sugden, In: Gene Transfer, Kucherlapati (Ed.), Plenum Press, NY, 117-148, 1986.
Bakhshi et al., Cell, 41(3):899-906, 1985.
Behbakht et al., Cancer Res., 67:3036-3042. 2007.
Bellus, J. Macromol. Sci. Pure Appl. Chem., A31(1): 1355-1376, 1994.
Boger et al., J. Am. Chem. Soc., 123(25):5878-5891, 2001.
Boutselis et al., J. Med. Chem., 50(4):856-864, 2007.
Brown et al., Immunol Ser, 53:69-82, 1990.
Bukowski et al., Clinical Cancer Res., 4(10):2337-2347, 1998.
Caldas et al., Cancer Res., 54:3568-3573, 1994.
Caldas et al., Nat. Genet., 8(1):27-32, 1994.
Capaldi et al., Biochem. Biophys. Res. Comm., 74(2):425-433, 1977.
Carbonelli et al., FEMS Microbiol. Lett., 177(1):75-82, 1999.
Chandler et al., Proc. Natl. Acad. Sci. USA, 94(8):3596-601, 1997.
Chen and Okayama, Mol. Cell Biol., 7(8):2745-2752, 1987.
Chene, Mol. Cancer Res., 2:20-28, 2004.
Cheng et al., Cancer Res., 54(21):5547-5551, 1994.
Christensen et al., Advances Cancer Res., 101:93-126, 2008.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cocea, Biotechniques, 23(5):814-816, 1997.
Coletta et al., Cancer Res., 68(7):2204-2213, 2008.
Coletta et al., Proc. Natl. Acad. Sci. USA, 101(17):6478-6483, 2004.
Coletta et al., Proc. Natl. Acad. Sci. USA, 101:6478-6483, 2004.
Coupar et al., Gene, 68:1-10, 1988.
Davidson et al., J. Immunother., 21(5):389-398, 1998.
De Jager et al., Semin. Nucl. Med., 23(2):165-179, 1993.
Doolittle and Ben-Zeev, Methods Mol, Biol, 109:215-237, 1999.
European Appln. 320 308
European Appln. 329 822
European Appln. 373 203
European Appln. 785 280
European Appln. 799 897
Fan et al., J. Biol. Chem., 275:32129-32134, 2000.
Fechheimer, et al., Proc Natl. Acad. Sci. USA, 84:8463-8467, 1987.
Fodor et al., Biochemistry, 30(33):8102-8108, 1991.
Ford et al., Proc. Natl. Acad. Sci. USA, 95:12608-126 1998.
Fraley et al., Proc. Natl. Acad. Sci. USA, 76:3348-3352, 1979.
Friedmann, Science, 244:1275-1281, 1989.
Frohman, In: PCR Protocols: A Guide To Methods And Applications, Academic Press, N.Y., 1990.
Garvie et al., Mol. Cell, 8:1267-1276, 2001.
GB Appln. 2 202 328
Gidekel et al., Cancer Cell, 4:361-370, 2003.
Gopal, Mol. Cell Biol., 5:1188-1190, 1985.
Graham and Van Der Eb, Virology, 52:456-467, 1973.
Gulbis and Galand, Hum. Pathol., 24(12):1271-1285, 1993.
Gunther et al., FASEB J., 16:283-292, 2002.
Hacia et al., Nature Genet., 14:441-449, 1996.
Hanibuchi et al., Int. J. Cancer, 78(4):480-485, 1998.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Hellstrand et al., Acta Oncologica, 37(4):347-353, 1998.
Hendrickson and Ogata, In: Methods in Enzymology, Carters and Sweet (Eds.), 276: 494-523, Academic Press, Inc., 1997.
Hermonat and Muzycska, Proc. Natl. Acad. Sci. USA, 81:6466-6470, 1984.
Horwich et al. J. Virol., 64:642-650, 1990.
Hovde et al., Biochemistry, 40:12013-12021, 2001.
Hui and Hashimoto, Infection Immun., 66(11):5329-5336, 1998.
Hussussian et al., Nat. Genet., 8(1):15-21, 1994.
Innis et al., Proc. Natl. Acad. Sci. USA, 85(24):9436-9440, 1988.
Inouye and Inouye, Nucleic Acids Res., 13:3101-3109, 1985.
Jemc and Rebay, Annu. Rev. Biochem., 76:513-538, 2007.
Johnson et al., In: Biotechnology and Pharmacy, Pezzuto et al., eds., Chapman and Hall, New York, 1993.
Ju et al., Gene Ther., 7(19):1672-1679, 2000.
Kaeppler et al., Plant Cell Reports 9: 415-418, 1990.
Kamb et al., Nat. Genet., 8(1):23-26, 1994.
Kamb et al., Science, 2674:436-440, 1994.
Kaneda et al., Science, 243:375-378, 1989.
Kato et al, J. Biol. Chem., 266:3361-3364, 1991.
Kawakami et al., Bioessays, 22(7):616-626, 2000.
Kawakami et al., Nucleic Acids Res., 24(2):303-310, 1996.
Kelly et al., Blood, 99:310-318, 2002.
Kern et al., Breast Cancer Res. Treat., 31:153-65, 1994.
Kerr et al., Br. J. Cancer, 26(4):239-257, 1972.
Khan et al., Proc. Natl. Acad. Sci. USA, 96(23):13264-13269, 1999.
Kurebayashi et al., Cancer Res., 53:2178-87, 1993.
Kwoh et al., Proc. Natl. Acad. Sci. USA, 86:1173, 1989.
Kyte and Doolittle, J. Mol. Biol., 157(1):105-132, 1982.
Laclef et al., Development, 130(10):2239-2252, 2003.
Laclef et al., Mech. Dev., 120(6):669-679, 2003. Ozaki et al., 2004
Lepourcelet et al., Cancer Cell, 5:91-102, 2004.
Levenson et al., Hum. Gene Ther., 9(8):1233-1236, 1998.
Li et al., Am. J. Pathol., 160(6):2181-2190, 2002.
Li et al., Nature, 426(6964):247-254, 2003.
MacBeath and Schreiber, Science, 289(5485):1760-1763, 2000.
Macejak and Sarnow, Nature, 353:90-94, 1991.
Mao et al., J. Biol. Chem., 283:12819-12830, 2008.
Merrifield, Science, 232(4748):341-347, 1986.
Minn et al., Proc. Natl. Acad. Sci. USA, 104:6740-6745, 2007.
Mitchell et al., Ann. NY Acad. Sci., 690:153-166, 1993.
Mitchell et al., J. Clin. Oncol., 8(5):856-869, 1990.
Moerke et al., Cell, 128:257-267, 2007.
Mori et al., Cancer Res., 54(13):3396-3397, 1994.
Morton et al., Arch. Surg., 127:392-399, 1992.
Myszka and Rich, Pharm. Sci. Technolo. Today, 3(9):310-317, 2000.
Nakamura et al., In: Handbook of Experimental Immunology (4^{th} Ed.), Weir et al., (eds). 1:27, Blackwell Scientific Publ., Oxford, 1987.
Ng et al., Br. J. Cancer, 95(8):1050-1055, 2006.
Nicolas and Rubinstein, In: Vectors: A survey of molecular cloning vectors and their uses, Rodriguez and Denhardt, eds., Stoneham: Butterworth, pp. 494-513, 1988
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Ohara et al., Proc. Natl. Acad. Sci. USA, 86:5673-5677, 1989.
Okamoto et al., Proc. Natl. Acad. Sci. USA, 91(23):11045-11049, 1994.
Olenyuk et al., Proc. Natl. Acad. Sci. USA, 101(48):16768-16773, 2004.
Olenyuk et al., Proc. Natl. Acad. Sci. USA, 101:16768-16773, 2004.
Oliver et al., Development, 121(3):693-705, 1995.1995
Omirulleh et al., Plant Mol. Biol., 21(3):415-428, 1993.
Orlow et al., Cancer Res, 54(11):2848-2851, 1994.
Orlow et al., Int. J. Oncol., 15(1):17-24, 1994.
Pandey and Mann, Nature, 405(6788):837-846, 2000.
PCT Appln. PCT/US87/00880
PCT Appln. PCT/US89/01025
PCT Appln. WO 0138580
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100012
PCT Appln. WO 03100448A1
PCT Appln. WO 04020085
PCT Appln. WO 04027093
PCT Appln. WO 04048933
PCT Appln. WO 09923256
PCT Appln. WO 09936760
PCT Appln. WO 88/10315
PCT Appln. WO 89/06700
PCT Appln. WO 90/07641.
PCT Appln. WO 93/17126
PCT Appln. WO 94/09699
PCT Appln. WO 95/06128
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
Pease et al., Proc. Natl. Acad. Sci. USA, 91:5022-5026, 1994.
Pelletier and Sonenberg, Nature, 334(6180):320-325, 1988.
Pietras et al., Oncogene, 17(17):2235-2249, 1998.
Piper et al., Cell, 96:587-597, 1999.
Potrykus et al., Mol. Gen. Genet., 199(2):169-177, 1985.
Qin et al., Proc. Natl. Acad. Sci. USA, 95(24):14411-14416, 1998.
Rayapureddi et al., Nature, 426(6964):295-298, 2003.
Ravindranath and Morton, Intern. Rev. Immunol., 7: 303-329, 1991.
Rayapureddi et al., Nature, 426(6964):295-298, 2003.
Reichenberger et al., Cancer Res., 65(7):2668-2675, 2005.
Ridgeway, In: Vectors: A Survey of Molecular Cloning Vectors and Their Uses, Rodriguez et al. (Eds.), Stoneham: Butterworth, 467-492, 1988.
Rippe, et al., Mol. Cell Biol., 10:689-695, 1990.
Rishi et al., Anal. Biochem., 340(2):259-271, 2005.
Rishi et al., Anal. Biochem., 340:259-271, 2005.
Rosenberg et al., Ann. Surg. 210(4):474-548, 1989.
Rosenberg et al., N. Engl. J. Med., 319:1676, 1988.
Ruf et al., Proc. Natl. Acad. Sci. USA, 101(21):8090-8095, 2004.
Sambrook et al., In: Molecular cloning, Cold Spring Harbor Laboratory Press, Cold Spring Harbor, N.Y., 2001.
Sambrook et al., In: Molecular cloning: a laboratory manual, 2^{nd} Ed., Cold Spring Harbor Laboratory Press, Cold Spring Harbor, N.Y., 1989.
Serrano et al., Nature, 366:704-707, 1993.
Serrano et al., Science, 267(5195):249-252, 1995.
Shoemaker et al., Nature Genetics, 14:450-456, 1996.
Spitz et al., Proc. Natl. Acad. Sci. USA, 95(24):14220-14225, 1998.
Spitz et al., Proc. Natl. Acad. Sci. USA, 95:14220-14225, 1998.
Stewart and Young, In: Solid Phase Peptide Synthesis, 2d. ed., Pierce Chemical Co., 1984.
Tam et al., J. Am. Chem. Soc., 105:6442, 1983.
Temin, In: Gene Transfer, Kucherlapati (Ed.), NY, Plenum Press, 149-188, 1986.
Tootle et al., Nature, 426(6964):299-302, 2003.
Tootle et al., Nature, 426:299-302, 2003.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
UK Appln. 8 803 000
Vassilev et al., Science, 303:844-848, 2004.
Vigneswaran et al., Hum. Pathol., 31:931-937, 2000.
Vinson, Sci STKE, pe23, 2005.
Walker et al., Nucleic Acids Res. 20(7):1691-1696, 1992.
Wong et al., Gene, 10:87-94, 1980.
Xu et al., Curr. Biol., 13(9):790-795, 2003.
Xu et al., Development, 129(13):3033-3044, 2002.
Xu et al., Nature, 425(6955): 316-21, 2003.
Yu et al., Cancer Res., 66:1982-1989, 2006.
Yu et al., Nat. Med., 10(2):175-181, 2004.
Zhang et al., Cancer Res., 65(3):925-932, 2005.
Zhang et al., J. Assoc. Res. Otolaryngol., 5(3):295-304, 2004.
Zhang et al., Oncogene, 15:2093-2108, 1997.
Zheng et al., Development, 130(17):3989-4000, 2003.

1. A method of treating cancer in a subject comprising administering to said subject an effective amount of an inhibitor of Eya2 phosphatase activity.

2. The method of claim 1, wherein the inhibitor of Eya2 phosphatase activity is a nucleic acid, a protein, a peptide or a small molecule.

3. The method of claim 2, wherein the inhibitor of Eya2 phosphatase activity is a nucleic acid.

4. The method of claim 3, wherein the nucleic acid encodes an antibody that binds Eya2.

5. The method of claim 1, wherein the inhibitor of Eya2 phosphatase activity is a non-selective inhibitor or a selective inhibitor.

6. The method of claim 5, wherein the inhibitor of Eya2 phosphatase activity is a non-selective inhibitor.

7. The method of claim 6, wherein the non-selective inhibitor is Na₂MoO₄, β-glycerophosphate, NaF, or Na₃VO₄.

8. The method of claim 5, wherein the inhibitor of Eya2 phosphatase activity is a selective inhibitor.

9. The method of claim 1, wherein the inhibitor is administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage.

10. The method of claim 9, wherein the inhibitor of Eya2 phosphatase activity is a nucleic acid, a protein, a peptide or a small molecule.

11. The method of claim 9, further comprising administering to said patient a second cancer therapy.

12. The method of claim 11, wherein said second cancer therapy is radiotherapy, immunotherapy, chemotherapy, hormonal therapy or gene therapy.

13. The method of claim 9, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

14. A method of identifying a candidate anti-cancer agent comprising:
(a) contacting a cell expressing Eya2 with a test substance; and
(b) assessing the effect of the test substance on Eya2 phosphatase activity,
wherein inhibition of the interaction of Eya2 phosphatase activity indicates that said test substance is a candidate anti-cancer agent.

15. The method of claim 14, wherein the candidate inhibitor is a nucleic acid, a protein, a peptide or a small molecule.

16. A method of inhibiting interaction of Six1 with Eya2 comprising contacting a cell expressing Six1 and Eya2 with an agent that inhibits binding of Six1 to Eya2.

17. The method of claim 16, wherein the inhibitor of the Six1/Eya2 interaction is a nucleic acid, a protein, a peptide or a small molecule.

18. The method of claim 17, wherein the nucleic acid encodes an antibody that binds Six1 or Eya2.

19. The method of claim 17, wherein the peptide is a Six1 peptide decoy that binds Eya2.

20. The method of claim 17, wherein the peptide is an Eya2 peptide decoy that binds Six1.

21. A method of inhibiting interaction of Six1 with Eya2 interaction comprising contacting a cell with an agent that inhibits the phostphatase activity of Eya2 on Six1.

22. A method of treating cancer in a subject comprising administering to said subject an effective amount of an inhibitor of Six1 interaction with Eya2.

23. The method of claim 22, wherein the inhibitor is administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage.

24. The method of claim 23, wherein the inhibitor of the Six1/Eya2 interaction is a nucleic acid, a protein, a peptide or a small molecule.

25. The method of claim 23, wherein the nucleic acid encodes an antibody that binds Six1 or Eya2.

26. The method of claim 23, wherein the peptide is a Six1 peptide decoy that binds Eya2.

27. The method of claim 23, wherein the peptide is an Eya2 peptide decoy that binds Six1.

28. The method of claim 23, further comprising administering to said patient a second cancer therapy.

29. The method of claim 28, wherein said second cancer therapy is radiotherapy, immunotherapy, chemotherapy, hormonal therapy or gene therapy.

30. The method of claim 22, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

31. A method of treating cancer in a subject comprising administering to said subject an effective amount of an agent that inhibits the phosphatase activity of Eya2 on Six1.

32. A method of identifying a candidate anti-cancer agent comprising:
(a) contacting a cell expressing Six1 and Eya2 with a test substance; and
(b) assessing the effect of the test substance on the interaction Six1 with Eya2,
wherein inhibition of the interaction of Six1 with Eya2 indicates that said test substance is a candidate anti-cancer agent.

33. The method of claim 32, wherein the candidate inhibitor is a nucleic acid, a protein, a peptide or a small molecule.

34. A method of identifying a candidate anti-cancer agent comprising:
(a) contacting a cell expressing Six1 and Eya2 with a test substance; and
(b) assessing the effect of the test substance on the phosphatase activity of Eya2 on Six1,
wherein inhibition of the phosphatase activity of Eya2 on Six1 indicates that said test substance is a candidate anti-cancer agent.

35. The method of claim 34, wherein the candidate inhibitor is a nucleic acid, a protein, a peptide or a small molecule.
